(19) United States
(12) Patent Application Publication
(10) Pub. No.: US 2026/0294225 A1
(43) Pub. Date: Oct. 1, 2026

(54) SAFETY CAP FOR MULTI-FUNCTIONAL SURGICAL SCOPE TIP

(71) Applicant: Jackson Medical Group Inc., Atlanta, GA (US)

(72) Inventors: James Rains, Atlanta, GA (US); Kamil Makhnejia, Atlanta, GA (US)

(21) Appl. No.: 19/633,356

(22) Filed: Mar. 30, 2026

Related U.S. Application Data
(60) Provisional application No. 63/779,838, filed on Mar. 28, 2025.

(51) Int. Cl. A61B 1/12; A61B 1/00

(52) U.S. Cl. CPC A61B 1/123; A61B 1/00032; A61B 1/00097; A61B 1/00137; A61B 1/127

(57) ABSTRACT

An example device includes a heater for a surgical scope including: a thermally insulative housing or environment defining a receptacle, an elongated internal chamber disposed in the housing and having a first end, a second end, and an internal surface defining a central internal volume to receive and retain a surgical scope tip for protection against thermal damage to surrounding thermally sensitive materials, a circuit configured to heat the elongated internal chamber, a transistor coupled to the circuit board or circuit component and operatively coupled to a battery to generate heat, wherein the device is compactly portable when affixed to the surgical scope tip to allow the surgical scope tip to be oriented freely while inserted in the safety cap device.

### CROSS-REFERENCE TO RELATED APPLICATIONS

This application claims the benefit of U.S. provisional patent application No. 63/779,838, filed on Mar. 28, 2025, and titled “SAFETY CAP FOR MULTI-FUNCTIONAL SURGICAL SCOPE TIP,” the disclosure of which is expressly incorporated herein by reference in its entirety.

### BACKGROUND

Endoscopy and laparoscopy (traditional and robotic) are widely used for diagnostic purposes (e.g., colonoscopy, bronchoscopy, pelvic examination, etc.) and therapeutic procedures (e.g., polypectomy, cholecystectomy, etc.). An endoscopy is a procedure performed to examine the structures inside your body up close. During an endoscopy, a surgical scope with a long, thin, lighted tube (endoscope) is placed inside the body of the patient to examine an organ or an area of interest. Laparoscopy is a minimally invasive surgical procedure that allows a surgeon to examine the organs and structures in the abdomen and pelvis using a surgical scope with a long, thin, lighted tube (laparoscope).

A surgical scope tip is the distal end of the surgical or imaging scope that contains a camera and light assembly and is used to see inside a patient's body. During use, tips are heated and cleaned to prevent fogging due to the difference in temperature between the body and the operating room, which could block the view of the surgical site and body structure. Tip warming and defogging devices are commercially available to maintain the surgical scope in an upright position to defog the surgical scope.

There is, nevertheless, a benefit to improving the safety of surgical scopes for endoscopy, laparoscopy, and the like.

### SUMMARY

An exemplary safety cap device and method are disclosed for a surgical scope for endoscopy, laparoscopy, and the like, configured to be affixed to the surgical scope tip and allow the surgical scope tip to be oriented freely in a sideways manner, as well as upright and upside down position, while inserted in the safety cap device having a compact internal housing that is small in size, lightweight, and having a height-to-width (or diameter) ratio between 1:1 and 5:1 that is ergonomically and optimally sized for a clinician's hand. The safety cap may have active heating elements or passive elements to provide defogging utility.

Existing commercially available defogging devices have large bases to receive a scope tip to maintain while heating the scope tip and the surgical scope body in a generally upright manner. During surgery, surgical covering/drape and gown covers over the patient to expose a surgical area on the patient, and instruments are frequently placed, intentionally as part of the operating procedure or otherwise, on the surgical covering and gowns over the patient. While sufficient for the placement of compact surgical objects, the surgical drape/covering and gown over the patient's body are unstable for a long, thin, lighted tube of the surgical scope that is cumbersome and bulky, particularly when not within the patient's body. Surgical drapes/coverings and gowns are made of thin, flammable paper, meltable polypropylene, or silk material and can readily burn and catch on fire when exposed to even moderate heat. As such, there is a risk of fire from the inadvertent or impromptu use of the surgical area on the patient when placing the scope tip. The lighting assembly of endoscopy and laparoscopy instruments typically emits high-intensity light that can create a natural heating zone at the focal point of the light rays, some distance from the scope tip. Indeed, while existing tip warmer devices are designed to maintain surgical scope in an upright position, the danger of surgical scope (connected to a power source) tipping, becoming disconnected from the defogging device and causing a thermal event due to the exposed scope tip is high due to the long, thin, lighted tube often being placed in an unstable manner on the surgical space.

In having a compact form factor for its housing that is designed to be affixed to the surgical scope tip to allow the surgical scope tip to be oriented freely and particularly in a side-ways manner, as well as upright position, and in other orientations, while inserted in the safety cap device, the exemplary safety cap device can be readily and beneficially placed over the surgical drape/covering and gowns over the patient that would prevent the surgical scope tip from coming into contact with the surgical drape/covering and gowns. The compact form factor is small to fit over the surgical scope tip like a wand tip (accommodating tips of different sizes) and lightweight to provide at least an 8× retention-to-weight (e.g., for a ~50 gram surgical scope tip that provides, e.g., 8×-20×, e.g., for 5 mm, 8 mm, 10 mm sized scope tips). The internal components may be configured and arranged to allow for a very small and compact form factor that is ergonomically and optimally sized for a clinician's hand, (e.g., around about 8 cm in length or height) and the like. The external housing of the surgical scope tip may have buffer spaces between the internal heating component and outside shell (external housing) to provide a thermal buffer for the chamber and heated internal components to retain heat within the housing, allowing continuous operation of the surgical scope tip over an extended period, e.g., 5 hours or at least 5 hours, during a surgery, while drawing on a small battery (to provide for a small form factor and weight). The compact form factor exemplary safety cap device may include an internal housing and elongated internal chamber disposed in the housing having an internal surface defining a central internal volume to receive and retain a surgical scope tip with an interior surface of the elongated chamber for liquid and/or sponge disposed in the internal volume to act as a scope warmer and prevent fogging of, and clean, the scope tips. The exemplary device captures the heat generated by the high-intensity light being emitted from the scope tip naturally and redirects the energy to the components inside the vessel (e.g., liquid and/or sponge) to create a heating effect in the chamber environment, thereby also heating the scope tip lens and surround scope shaft.

The exemplary safety cap device and method may employ an optimized electric heating circuit and controls to facilitate rapid heating of the internal components to ready it for use in less than 5 minutes, while in combination with an optimized thermal insulated structure, to operate at an optimal temperature, e.g., around 46-48° C. or alternatively or additionally 48-52° over an extended period, e.g., 5 hours or at least 5 hours, during a surgery, while drawing on a small battery (again, to provide for a small form factor and weight). The optimization of the circuit and thermal insulated structure was observed to extend the period of heating operation of the exemplary safety cap device and method by over 2× over initial baseline designs without such optimizations (e.g., in the circuit, component selection).

The elongated internal chamber is configured to house the cleaning fluid or a sponge, to retain the cleaning fluid, and has a covering configured to conformally and frictionally receive the surgical scope tip and maintain a seal with the surgical scope tip that maintains the cleaning fluid in the space in the elongated thermally insulative chamber when the internal housing is oriented sideways or partially or fully upside down.

The exemplary safety cap device is configured, via its shape and internal weighting, to be compactly portable, lightweight, and properly weighted, to affix to the scope tip to allow the scope tip to be oriented freely and lay to rest in a side-ways manner, preventing the surgical scope tip from coming in contact with the surgical drape/covering and gowns, the insertion placing the safety cap device as a wand tip to the scope rather than as a base structure that tries to maintain the scope tip in an upright position. The exemplary safety cap device has a base that is configured to maintain the safety cap device in a stable manner when not inserted onto the surgical scope tip to maintain the cleaning fluid in the space but not impede the surgical scope from being placed in a side-ways or partially upside-down orientation. The exemplary device is configured with a small size and lightweight structure to allow for the ready placement and position over the surgical scope tip to maximize the surgical scope utilization and minimize operating room disruption. Scope tips are generally inserted into various positions of the body; the exemplary safety device for the scope tips has a compact and small-sized design so as not to impede the activity of the surgical team while improving the safety of the operating room.

In some embodiments, the exemplary safety cap device has a heating element configured to provide rapid heating (e.g., ramp-up temperature to at least 40° C. in less than 5 minutes) and maintain an optimal temperature for an entire surgery. The exemplary safety cap device is configured with a built-in reservoir containing a cleaning solution to decontaminate the scope tips during or after surgery. In some embodiments, the exemplary system can draw power from a battery.

A surgical scope tip in operating rooms can have a long wire/cable connecting to a power source. The current state-of-the-art safety defogging devices are large and only configured to receive a scope tip and heat the scope tip in an upright manner. With the long wire/cable, when inserted in the current state-of-the-art defogging device, the heated scope tip can fall over and cause fire when the heated tip is in contact with a flammable material (e.g., skin, organs, silk). The exemplary safety cap device is configured to be compactly portable when affixed to the scope tip to allow the scope tip to be oriented freely in an upright or sideways manner while inserted in the safety cap device, preventing the scope tip from falling over (due to long wire/cable) and causing fire.

In an aspect, a safety cap device for a surgical scope tip (e.g., for an endoscope, surgical telescope, laparoscopic, robotic scope) is disclosed comprising a thermally insulative housing; and an elongated internal chamber disposed in the housing and having a first end, a second end, and an internal surface defining a central internal volume to receive and retain a surgical scope tip for protection against thermal damage to surrounding thermally sensitive materials, wherein the safety cap device defines a shape having a height-to-width (or diameter) ratio between 1:1 and 25:1 (e.g., between 1:1 and 5:1), and wherein the safety cap device is compactly portable when affixed to the surgical scope tip to allow the surgical scope tip to be oriented freely while inserted in the safety cap device.

In some embodiments, the safety cap device described herein further comprises a heating element (e.g., heating coil, or heating sheet) disposed on and in substantial thermal communication with an exterior surface of the elongated chamber for warming a liquid or sponge disposed in the internal volume (substantial=spanning more than 50% of area of exterior surface of the elongated chamber).

In some embodiments, the safety cap device described herein further comprises an electric circuit in electric communication with the heating element, wherein the first end includes one or more cover sealing members defining a device opening to receive and seal the surgical scope tip, wherein the second end is in contact with, proximal to, or surrounded by a battery holder housing at least one battery, and wherein the elongated chamber has a cross-section corresponding to the opening to define a gap to an exterior surface of the surgical scope tip when the surgical scope tip is disposed in the internal volume.

In some embodiments, the heating element is encapsulated in a thermal insulating layer (e.g., insulating sheet or form) in the internal volume.

In some embodiments, the safety cap device described herein further comprises an electric circuit disposed in the central internal volume of the internal chamber, wherein the first end includes one or more cover sealing members defining a device opening to receive and seal the surgical scope tip, wherein the second end is in contact with, proximal to, or surrounded by a battery holder housing at least one battery, and wherein the elongated chamber has a cross-section corresponding to the opening to define a gap to an exterior surface of the surgical scope tip when the surgical scope tip is disposed in the internal volume.

In some embodiments, the housing defines (i) a first section to house the elongated internal chamber and heating element and (ii) a second section, as a battery holder, to house the at least one battery, the second end of the elongated internal chamber in contact with a housing wall defined between the first section and the second section.

In some embodiments, the housing defines (i) a first section to house the elongated internal chamber and (ii) a second section, as a battery holder, to house the at least one battery, the second end of the elongated internal chamber in contact with a housing wall defined between the first section and the second section.

In some embodiments, the first section is configured with a reflective coating.

In some embodiments, the first section is configured with a ceramic coating.

In some embodiments, the elongated internal chamber is cylindrical.

In some embodiments, the elongated internal chamber has a shape in correspondence to an exterior surface of the surgical scope tip.

In some embodiments, the battery has a center axis corresponding to an elongated axis of the elongated internal chamber.

In some embodiments, the battery has a center axis perpendicular to an elongated axis of the elongated internal chamber.

In some embodiments, the battery has a center axis parallel to an elongated axis of the elongated internal chamber.

In some embodiments, the battery is disposed proximal to the surgical scope tip (i.e., top of the safety cap device).

In some embodiments, the internal housing has (i) a bullet shape, (ii) a cylindrical shape, (iii) an inkwell shape, or (iv) a nose cone shape having a height-to-width (or diameter) ratio between 1:1 and 25:1.

In some embodiments, the electric circuit includes a thermoswitch disposed on a surface of the heating element.

In some embodiments, the electric circuit includes a thermoswitch configured to break electrical contact between the battery and the heating element when the thermoswitch exceeds a predefined temperature threshold having a correspondence to a maximum temperature for the internal volume (e.g., between 40° C. and 55° C.).

In some embodiments, the electric circuit includes a contact switch configured to make electrical contact between the battery and the heating element when the surgical scope tip is disposed in the internal volume and in contact with the contact switch.

In some embodiments, the electric circuit includes a toggle, pull tab, or switch configured to make electrical contact between the battery and the heating element.

In some embodiments, the electric circuit includes a voltage regulator to boost the voltage output of the battery and a pulse-width modulator (PWM) to regulate the power of the battery.

In some embodiments, the battery is sized with energy to rapidly heat the central internal volume in combination with heat from the surgical scope tip.

In some embodiments, the one or more cover sealing members include a first compliant cover sealing member that couples to an orifice defining the first end of the elongated internal chamber to define the device opening (e.g., the first compliant cover sealing member having a snap member that extends into a portion of the elongated internal chamber to couple therewith).

In some embodiments, the one or more cover sealing members include a second compliant cover sealing member that couples to the first compliant cover sealing member, the first compliant cover sealing member and the second compliant cover sealing member each having a respective orifice to define the device opening.

In some embodiments, the first compliant cover sealing member has a second orifice (e.g., to allow air venting during surgical scope tip insertion into the elongated internal chamber and through the first compliant cover sealing member).

In another aspect, a method is disclosed comprising the steps of using the above-discussed safety cap device.

In another aspect, a device is disclosed for a surgical scope tip (e.g., for an endoscope, surgical telescope, laparoscopic, robotic scope) comprising: a thermally insulative housing defining a receptacle opening on a top surface and configured to receive a surgical scope tip; an elongated internal chamber disposed in the housing and having a first end, a second end, and an internal surface defining a central internal volume to receive and retain a surgical scope tip for protection against thermal damage to surrounding thermally sensitive materials; a circuit board or circuit component (e.g., heating circuit) (e.g., printed circuit board or flexible circuit) disposed to (or around) a bottom surface of the elongated internal chamber distal and below the receptacle opening (e.g., wherein the circuit board or circuit component includes at least one thermal conducting via, thermal conducting bus, wire, pad); and a transistor coupled to the circuit board or circuit component and operatively coupled to a battery to generate heat (e.g., wherein the transistor is configured to heat the receptacle through the at least one thermal via), wherein the device defines a shape having a height-to-width (or diameter) ratio between 1:1 to 5:1 (e.g., about 2.4 or 1.36), and wherein the device is compactly portable when affixed to the surgical scope tip to allow the surgical scope tip to be oriented freely while inserted in the safety cap device.

In some embodiments, the circuit board or circuit component includes a sensor disposed on a second side of the printed board, wherein the sensor is positioned at a side of, to be in thermal contact with, the elongated internal chamber.

In some embodiments, the circuit board or circuit component has a surface area generally conforming to a bottom portion of the elongated internal chamber.

In another aspect, a device is disclosed for a surgical scope tip comprising: a first valve seal defining a first valve seal opening; a second valve seal defining a second valve seal opening; and a housing defining a housing opening having a first rib and a second rib, wherein the first and second rib are configured to retain the first valve seal and second valve seal, wherein the first valve seal and second valve seal are disposed between the first rib and the second rib so that the first valve seal opening, second valve seal opening, and housing opening are substantially coaxial, and wherein the first and second rib are configured to apply compression to the first valve seal and the second valve seal, wherein the device defines a shape having a height-to-width (or diameter) ratio between 1:1 and 5:1 (e.g., about 2.4 or 1.36), and wherein the device is compactly portable when affixed to the surgical scope tip to allow the surgical scope tip to be oriented freely while inserted in the safety cap device. The valve or valves may sit between two surfaces, e.g., via a rib. In other embodiments, a retaining ring (C-clip) may be employed to act as a surface to hold the valve or valves in place.

In some embodiments, the device includes a receptacle with a first end and a second end, wherein the first end comprises an opening and a retaining lip, and wherein the retaining lip is configured to be positioned between the first rib and second rib of the housing (e.g., wherein the first valve seal and the second valve seal are configured to provide at least 8× retention to the weight of the device (e.g., 50 grams) (e.g., 8×-20×, e.g., for 5 mm, 8 mm, 10 mm sized scope tips)

In some embodiments, the device includes an elongated internal chamber disposed in the housing and having a first end, a second end, and an internal surface defining a central internal volume to receive and retain a surgical scope tip for protection against thermal damage to surrounding thermally sensitive materials; and a circuit board or circuit component disposed to (or around) a bottom surface of the elongated internal chamber distal and below the receptacle opening, wherein the circuit board or circuit component comprises a transistor coupled to the circuit board or circuit component and operatively coupled to a battery to generate heat.

In another aspect, a device is disclosed for a surgical scope tip comprising a housing defining a receptacle opening configured to receive a surgical scope tip, and a bottom surface configured to support the housing in a vertical orientation, wherein the housing defines a vertical dimension and a horizontal dimension, wherein the vertical dimension is defined by a distance between the opening and the bottom surface, and wherein the horizontal dimension is perpendicular to the vertical dimension, and wherein the vertical dimension is between 1.3 and 2.4 times larger than the horizontal dimension (e.g., having a ratio around 1.3 and 1.4 or a ratio around 2.4); and a battery disposed at a bottom portion inside the housing.

In another aspect, a device is disclosed for a surgical scope tip (e.g., for an endoscope, surgical telescope, laparoscopic, robotic scope) comprising: an electric circuit disclosed on a surface of the elongated internal chamber, the electric circuit comprising (i) a transistor and (ii) a thermal sensor, wherein the thermal sensor operatively couples, as a voltage divider circuit, to the transistor to drive the modulation of the transistor at low voltage at less 3.3V, wherein modulation or current flow through the transistor generates heat, and wherein the thermal sensor in the voltage divider circuit provide feedback control to the transistor to maintain a temperature of the thermal sensor (or the elongated internal chamber) at a pre-defined temperature (e.g., wherein the device defines a shape having a height-to-width (or diameter) ratio between 1:1 and 25:1, and wherein the device is compactly portable when affixed to the surgical scope tip to allow the surgical scope tip to be oriented freely while inserted in the safety cap device)

In another aspect, a device (e.g., safety cap device) is disclosed for a surgical scope tip (e.g., for an endoscope, surgical telescope, laparoscopic, robotic scope) comprising: a thermally insulative housing; and an elongated internal chamber disposed in the housing and having a first end, a second end, and an internal surface defining a central internal volume to receive and retain a surgical scope tip for protection against thermal damage to surrounding thermally sensitive materials, wherein the safety cap device defines a shape having a height-to-width (or diameter) ratio between 1:1 and 5:1, and wherein the safety cap device is compactly portable when affixed to the surgical scope tip to allow the surgical scope tip to be oriented freely while inserted in the safety cap device.

In some embodiments, the device includes a heating element (e.g., heating coil, or heating sheet) disposed on and in substantial thermal communication with an exterior surface of the elongated chamber for warming a liquid or sponge disposed in the internal volume (e.g., wherein substantial refers to spanning more than 50% of area of exterior surface of the elongated chamber).

In some embodiments, the device includes an electric circuit in electric communication with the heating element, wherein the first end comprises one or more cover sealing members defining a device opening to receive and seal the surgical scope tip, wherein the second end is in contact with, proximal to, or surrounded by a battery holder housing at least one battery, and wherein the elongated chamber has a cross-section corresponding to the opening to define a gap to an exterior surface of the surgical scope tip when the surgical scope tip is disposed in the internal volume.

In some embodiments, the heating element is encapsulated in a thermal insulating layer (e.g., insulating sheet or form) in the internal volume

In some embodiments, the device includes an electric circuit disposed in the central internal volume of the internal chamber, wherein the first end comprises one or more cover sealing members defining a device opening to receive and seal the surgical scope tip, wherein the second end is in contact with, proximal to, or surrounded by a battery holder housing at least one battery, and wherein the elongated chamber has a cross-section corresponding to the opening to define a gap to an exterior surface of the surgical scope tip when the surgical scope tip is disposed in the internal volume.

In some embodiments, the housing defines (i) a first section to house the elongated internal chamber and heating element and (ii) a second section, as a battery holder, to house the at least one battery, the second end of the elongated internal chamber in contact with a housing wall defined between the first section and the second section.

In some embodiments, the housing defines (i) a first section to house the elongated internal chamber and (ii) a second section, as a battery holder, to house the at least one battery, the second end of the elongated internal chamber in contact with a housing wall defined between the first section and the second section.

In some embodiments, the elongated internal chamber is cylindrical or oval.

In some embodiments, the elongated internal chamber has a shape in correspondence to an exterior surface of the surgical scope tip.

In some embodiments, the battery has a center axis corresponding to an elongated axis of the elongated internal chamber.

In some embodiments, the battery has a center axis perpendicular to an elongated axis of the elongated internal chamber.

In some embodiments, the battery has a center axis parallel to an elongated axis of the elongated internal chamber.

In some embodiments, the battery is disposed proximal to the surgical scope tip (i.e., top of the safety cap device).

In some embodiments, the external housing has (i) a bullet shape, (ii) a cylindrical shape, (iii) an inkwell shape, or (iv) a nose cone shape having a height-to-width (or diameter) ratio between 1:1 and 5:1.

In some embodiments, the electric circuit comprises a thermoswitch disposed on a surface of the heating element (e.g., employed as a thermal regulator). In some embodiments, the electric circuit comprises a thermoswitch configured to break electrical contact between the battery and the heating element when the thermoswitch exceeds a predefined temperature threshold having a correspondence to a maximum temperature for the internal volume (e.g., between 40° C. and 55° C.).

In some embodiments, the electric circuit comprises a thermistor or thermocouple configured to control the heating element.

In some embodiments, the electric circuit comprises a contact switch configured to make electrical contact between the battery and the heating element when the surgical scope tip is disposed in the internal volume and in contact with the contact switch.

In some embodiments, the electric circuit comprises a toggle, pull tab or switch configured to make electrical contact between the battery and the heating element.

In some embodiments, the electric circuit comprises a voltage regulator to boost voltage output of the battery.

In some embodiments, the battery is sized with energy to rapidly (e.g., within 4-5 minutes) heat the central internal volume in combination with heat from the surgical scope tip.

In some embodiments, the one or more cover sealing members include a first compliant cover sealing member that couples to an orifice defining the first end of the elongated internal chamber to define the device opening (e.g., the first compliant cover sealing member having a snap member that extends into a portion of the elongated internal chamber to couple therewith).

In some embodiments, the one or more cover sealing members include a second compliant cover sealing member that couples to the first compliant cover sealing member, the first compliant cover sealing member and the second compliant cover sealing member each having a respective orifice to define the device opening.

In some embodiments, the first compliant cover sealing member has a second orifice (e.g., to allow air venting during surgical scope tip insertion into the elongated internal chamber and through the first compliant cover sealing member).

In another aspect, a method is disclosed comprising: inserting a surgical scope tip into a housing of a device according to any one of the above-discussed features; defogging or cleaning the surgical scope tip; and regulating temperature in the receptacle.

In some embodiments, the method includes cleaning the surgical scope tip comprises applying a foam to the surgical scope tip.

In some embodiments, the device comprises a vent aperture, and inserting the surgical scope tip comprises venting air through the vent aperture.

### BRIEF DESCRIPTION OF THE DRAWINGS

The components in the drawings are not necessarily to scale relative to each other. Like reference numerals designate corresponding parts throughout the several views.

FIG. 1A-1D each illustrates a block diagram of an example implementation of the present disclosure.

FIG. 2 illustrates an example method of operation for the surgical scope heaters and caps disclosed herein.

FIG. 3A illustrates a cross-sectional view of an example safety cap for a surgical scope tip according to embodiments of the present disclosure.

FIG. 3B illustrates an exploded view of the receptacle, first valve seal, and second valve seal of FIG. 3A.

FIG. 3C illustrates an example circuit board used in the example embodiment of FIG. 3A.

FIG. 3D illustrates an exterior perspective view of a first side of the housing shown in FIG. 3A.

FIG. 3E illustrates an exterior perspective view of a second side of the housing shown in FIG. 3A.

FIG. 3F illustrates an exterior view of a top side of the housing shown in FIG. 3A.

FIG. 3G illustrates an example implementation of the present disclosure, including a battery and receptacle mounted so that the receptacle is offset horizontally from the battery.

FIG. 3H illustrates a block diagram of an example implementation of the present disclosure, including a battery and receptacle positioned so that the receptacle is mounted above the battery.

FIG. 4. Illustrates an example housing design configured to house two coin cell batteries (e.g., CR2477-sized batteries).

FIG. 5 illustrates an example housing design with a bullet-shaped profile, and configured to house two batteries (e.g., CR123-sized batteries).

FIG. 6 illustrates an example housing design configured to house two coin cell batteries (e.g., CR2477-sized batteries) with rounded edges.

FIG. 7 illustrates an example housing design configured with an “inkwell” shape with rounded edges and configured to house two batteries (e.g., CR123-sized batteries)

FIG. 8 illustrates another example housing design configured with an “inkwell” shape with rounded edges and configured to house two batteries (e.g., CR123-sized batteries).

FIG. 9 illustrates an example housing with a nose-cone design and configured to house two batteries (CR123)

FIG. 10 illustrates an example embodiment of the present disclosure compared to conventional devices.

FIG. 11 illustrates additional example housing shapes that can be used in embodiments of the present disclosure.

FIG. 12 illustrates a plot of the temperature of the surgical scope tip in (i) active heating (i.e., powered warming) using a heat element and (ii) passive heating (i.e., recaptured heating).

FIG. 13 illustrates a comparison of temperature vs. time for an example embodiment of the present disclosure compared to a conventional device.

FIG. 14 illustrates a comparison of temperature vs. time for an example embodiment of the present disclosure compared to a conventional device.

FIG. 15 illustrates an example computing device.

FIG. 16 illustrates an example valve assembly that can be used with the example implementations of the present disclosure described herein

FIG. 17 illustrates an example circuit design configured for a form factor capable of multiple orientations, according to implementations of the present disclosure.

FIG. 18 illustrates an example circuit design for fast ramping of temperature up to a desired temperature for scope warming, according to implementations of the present disclosure.

### DETAILED DESCRIPTION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entirety and to the same extent as if each reference were individually incorporated by reference.

### Example Systems

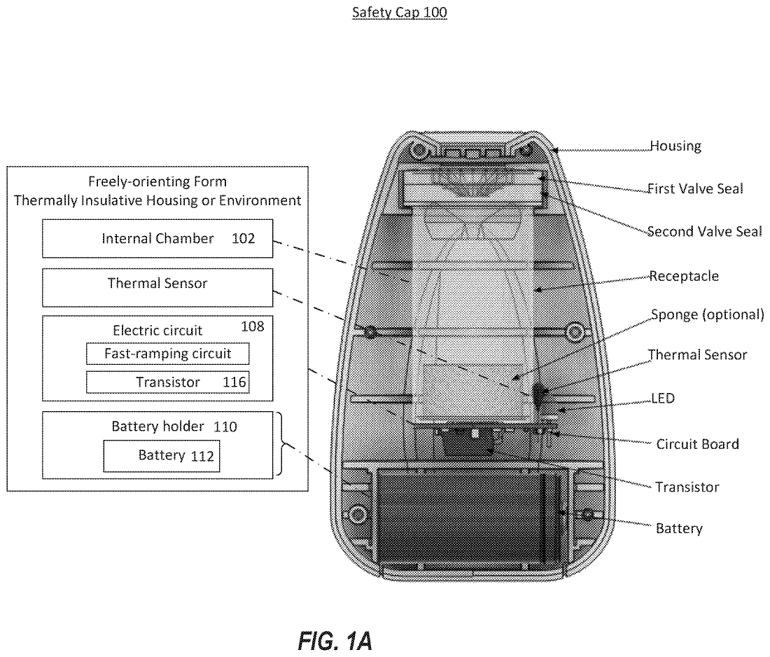

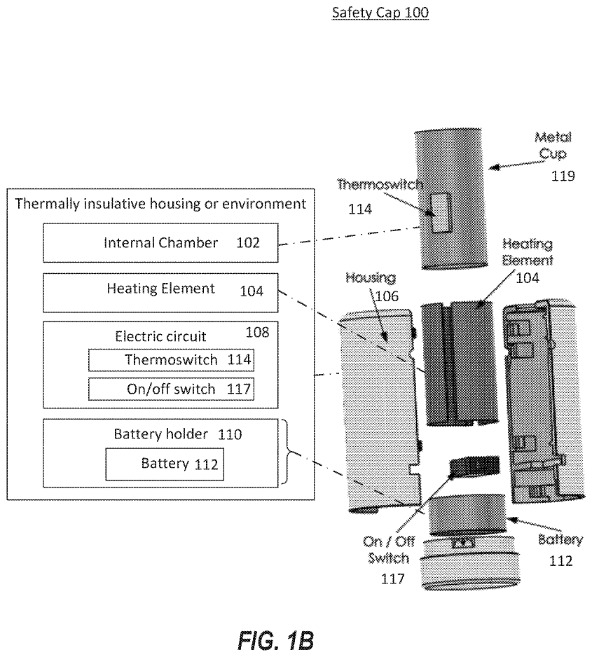

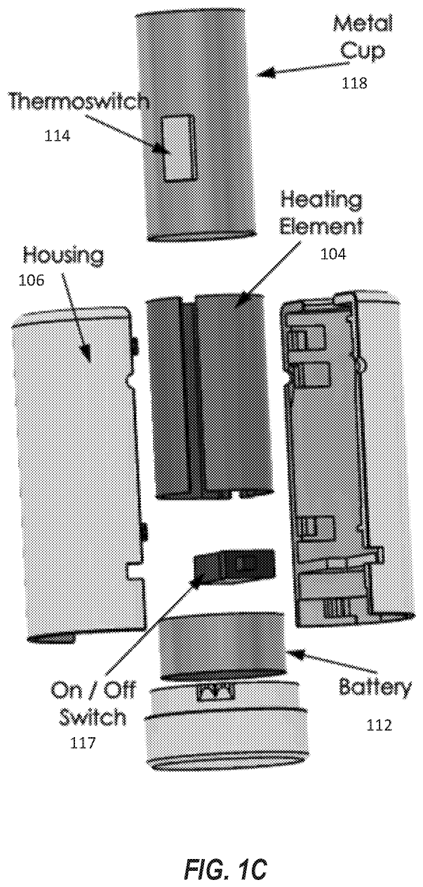

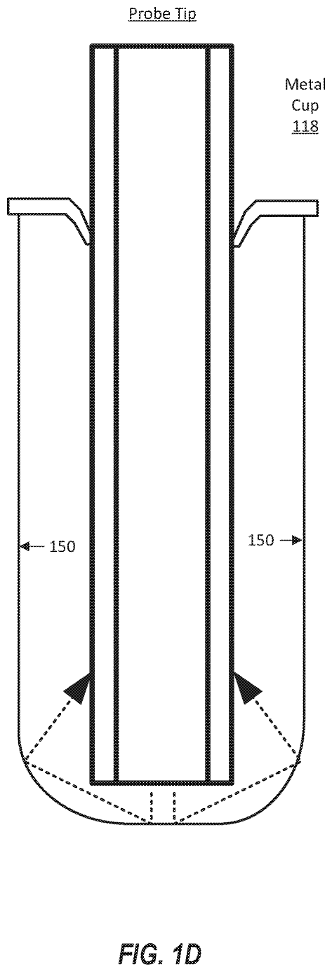

Example System #1. FIG. 1A shows an example safety cap 100 for a surgical scope tip. The example safety cap 100 includes a thermally insulative housing 106 defining a receptacle opening configured to receive a surgical scope tip, an elongated internal chamber 102 disposed in the housing 106 to receive and retain a surgical scope tip for protection against thermal damage to surrounding thermally sensitive materials, a circuit board or circuit component 108 (e.g., heating circuit) (e.g., printed circuit board or flexible circuit) disposed to (or around) a bottom surface of the elongated internal chamber distal and below the receptacle opening (e.g., wherein the circuit board or circuit component includes at least one thermal conducting via, thermal conducting bus, wire, pad), and a transistor 116 coupled to the circuit board or circuit component and operatively coupled to a battery to generate heat (e.g., wherein the transistor is configured to heat the receptacle through the at least one thermal via). The device defines a shape having a height-to-width (or diameter) ratio between 1:1 to 5:1 (e.g., between 1.3 and 1.4 or around 2.4). The device is compact and portable when affixed to the surgical scope tip, allowing the tip to be oriented freely while inserted into the safety cap device. The safety cap 100 can fit over the surgical scope tip like a wand tip and is lightweight to provide at least an 8× retention-to-weight that enables different orientations of the surgical scope tip, so that the surgical scope tip and safety cap 100 can be freely oriented in space. The housing can further be a thermally-insulative housing or environment where the housing structure or the internal air buffer in the structure provides thermal insulation to the internal chamber 102 and the heating components attached thereto.

Electric circuit 108 may include a transistor 116 and a fast-ramping circuit 117 to provide a heating circuit, for example, by a printed circuit board or flexible circuit and can be disposed to (or around) a bottom surface of the elongated internal chamber distal and below the receptacle opening (e.g., the circuit board or circuit component includes at least one thermal conducting via, thermal conducting bus, wire, pad). The heating circuit may be implemented using a transistor and a resistor network configured to provide heating.

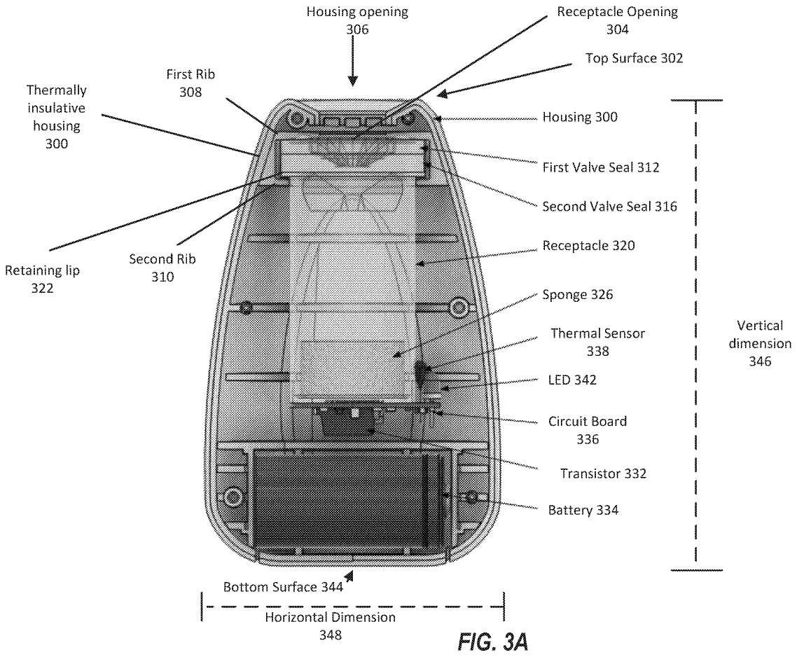

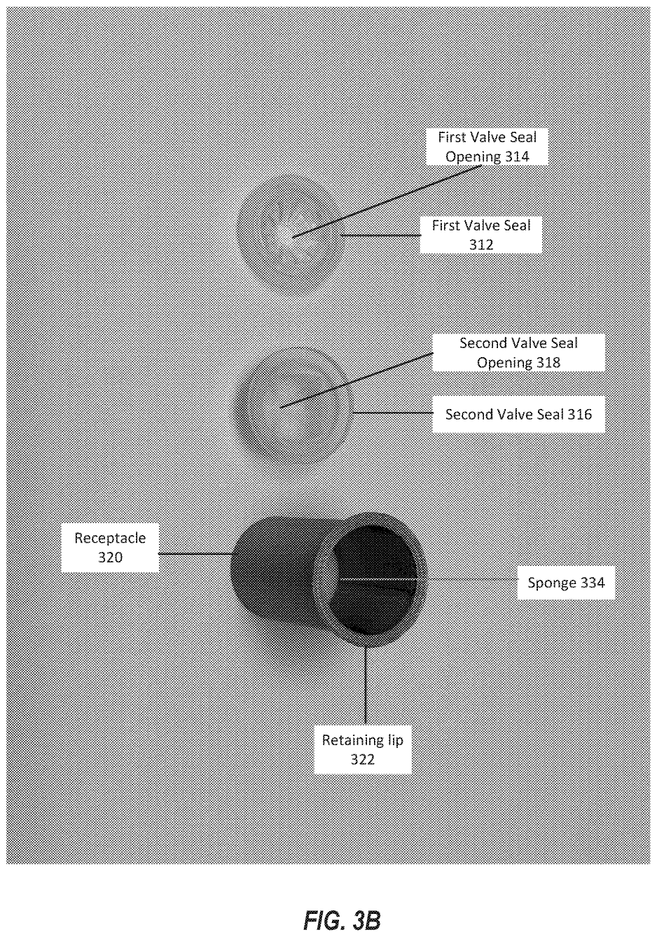

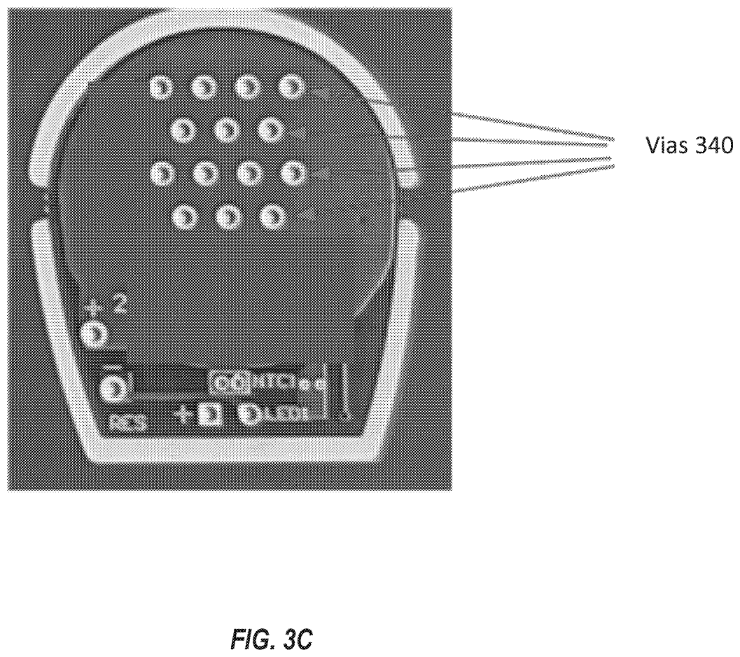

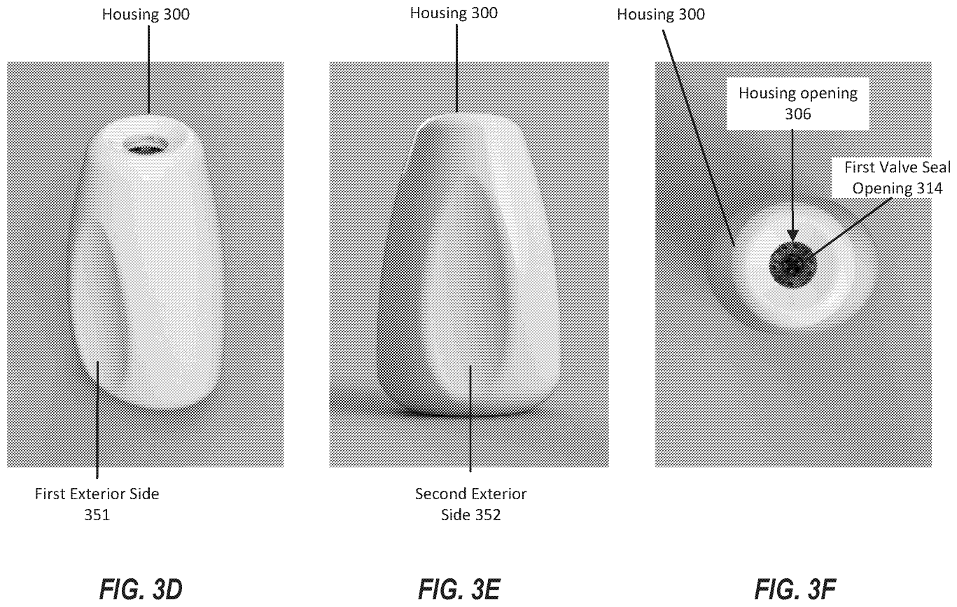

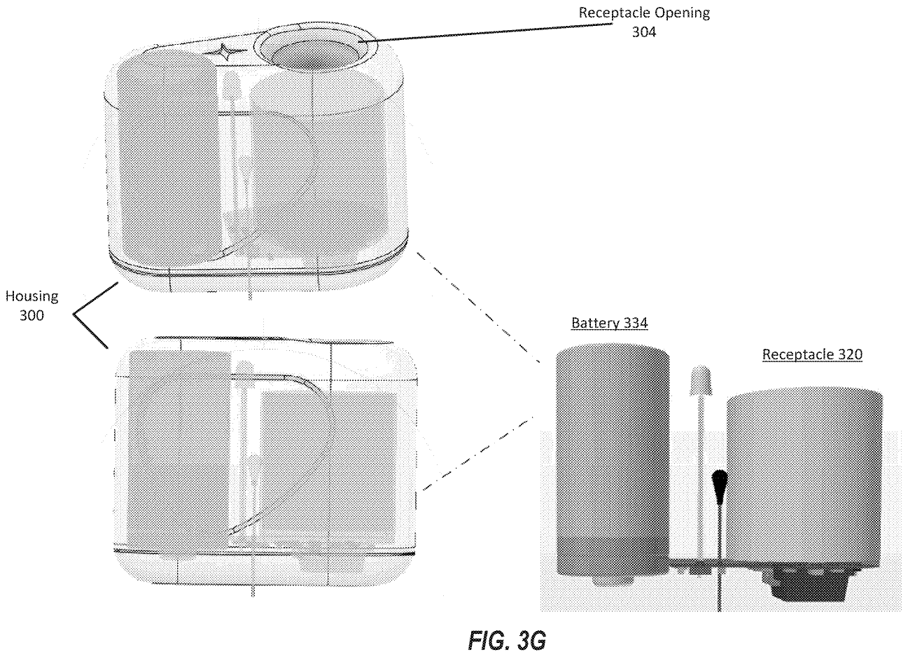

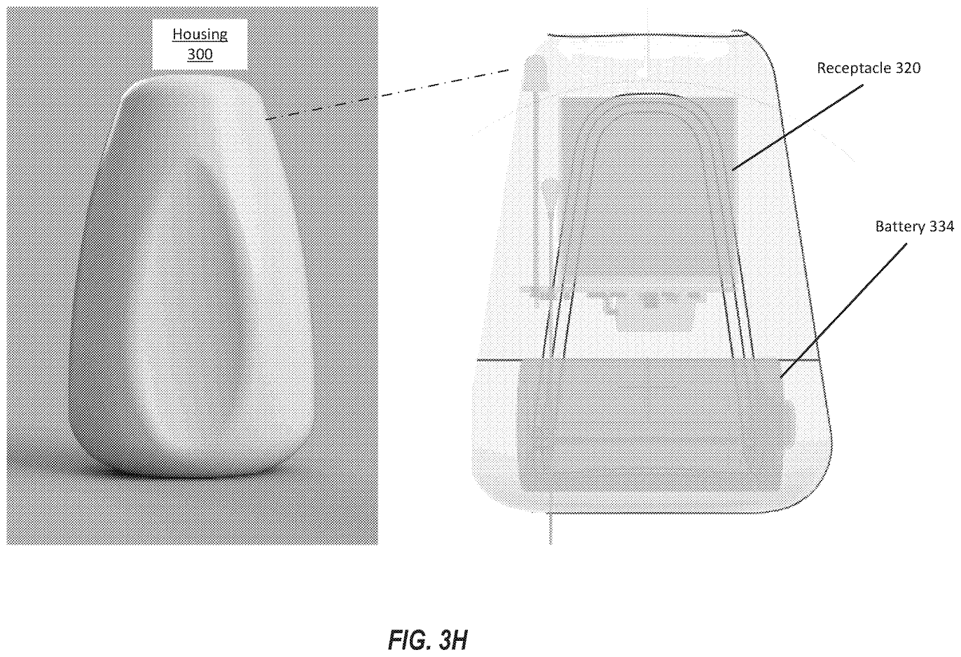

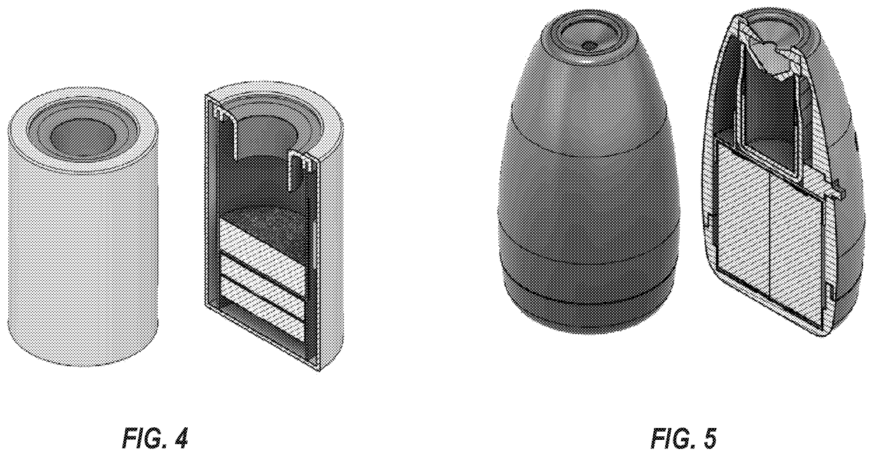

In the example shown in FIG. 1A, the safety cap 100 includes a battery holder 110 with a battery 112 that positions the battery within the internal chamber 102. The safety cap device 100 shown in FIG. 1A is shown in relation to a device shown in FIG. 3A, though device 100 may be applied to other devices and form factors described herein, e.g., shown in FIGS. 4-11.

The housing can include an internal chamber 102 (e.g., the receptacle described with reference to FIG. 3A. The internal chamber 102 can be an elongated internal chamber and can be disposed in the housing. The internal chamber 102 can define a first end, a second end, and an internal surface defining a central internal volume to receive and retain a surgical scope tip for protection against thermal damage to surrounding thermally sensitive materials. The housing may include a first section to house the elongated internal chamber 102 and heating element 116 and (ii) a second section, as a battery holder, to house the at least one battery. The sections may be separated by a wall or a partial wall. The elongated internal chamber (e.g., 102) may be cylindrical, oval, or other receptacle shaped. In some embodiments, the elongated internal chamber (e.g., 102) has a shape in correspondence to an exterior surface of the surgical scope tip. The battery 112 may have a center axis corresponding to an elongated axis of the elongated internal chamber. The battery 112 may be disposed proximal to the surgical scope tip (i.e., top of the safety cap device).

As described throughout the present disclosure, the device housing can define a shape with a height-to-width ratio configured to make the device compactly portable when affixed to the surgical scope tip to allow the surgical scope tip to be freely oriented while inserted in the safety cap. Non-limiting examples of height-to-width ratios that can be used include 5:1 through 1:1. The compact form factor is small to fit over the surgical scope tip like a wand tip (accommodating tips of different sizes) and lightweight to provide at least an 8× retention-to-weight (e.g., for a ~50 gram surgical scope tip that provides, e.g., 8×-20×, e.g., for 5 mm, 8 mm, 10 mm sized scope tips). The internal components may be configured and arranged to allow for a very small and compact form factor that is ergonomically and optimally sized for a clinician's hand, e.g., around about 8 cm and the like.

The circuit board or circuit component (e.g., 108) includes a sensor 109 (e.g., thermal sensor) disposed on a side of the printed board, wherein the sensor 109 is positioned at a side of, to be in thermal contact with, the elongated internal chamber 102. In some embodiments, the sensor 109 may be a thermistor, a thermoswitch, a thermocouple, a resistance temperature detector (RTD), a bimetallic strip, or the like. The circuit board or circuit component has a surface area generally conforming to the bottom portion of the elongated internal chamber.

Notably, the housing 106 defines a vertical dimension and a horizontal dimension, wherein the vertical dimension is defined by a distance between the opening and the bottom surface, and wherein the horizontal dimension is perpendicular to the vertical dimension, and wherein the vertical dimension is between 1.3 and 2.4 times larger than the horizontal dimension. external housing may have (i) a bullet shape, (ii) a cylindrical shape, (iii) an inkwell shape, (iv) a nose cone shape, or other shapes described or shown herein.

Transistor 116 (or a heating coil, or heating sheet) as a heating element may be disposed on and in substantial thermal communication with an exterior surface of the elongated chamber 102 for warming a liquid or sponge disposed in the internal volume. The electric circuit 108 may be in electric communication with the heating element 104. The heating element 104 or chamber 102 may be encapsulated in a thermal insulating layer, e.g., an insulating sheet or foam.

The device 100 includes one or more cover sealing members, e.g., a first compliant cover sealing member that couples to an orifice defining the first end of the elongated internal chamber to define the device opening (e.g., the first compliant cover sealing member having a snap member that extends into a portion of the elongated internal chamber to couple therewith). The one or more cover sealing members may include a second compliant cover sealing member that couples to the first compliant cover sealing member, the first compliant cover sealing member and the second compliant cover sealing member each having a respective orifice to define the device opening. The first compliant cover sealing member may have a second orifice (e.g., to allow air venting during surgical scope tip insertion into the elongated internal chamber and through the first compliant cover sealing member). The cover sealing member can provide retaining force for one or more scope tip sizes.

The compact form factor is small to fit over the surgical scope tip like a wand tip (accommodating tips of different sizes) and lightweight to provide at least an 8× retention-to-weight (e.g., for a ~50 gram surgical scope tip that provides, e.g., 8×-20×, e.g., for 5 mm, 8 mm, 10 mm sized scope tips). The internal components may be configured and arranged to allow for a very small and compact form factor that is ergonomically and optimally sized for a clinician's hand, e.g., around about 8 cm and the like.

The example safety cap 100 is preferably designed for single use as a disposable device.

Example System #2. FIG. 1B shows an example safety cap 100 (shown as 100b) for a surgical scope tip (e.g., for an endoscope, surgical telescope, or laparoscopic scope, or robotic scope) comprising a thermally insulative housing 106, an elongated internal chamber 102 (e.g., thermally conductive chamber or thermally non-conductive chamber, shown as metal cup 119), a heating element 104, an electric circuit 108, and/or a battery holder 110 configured to hold a battery 112, in accordance with an illustrative embodiment. The example safety cap 100b is preferably designed for single use as a disposable device.

Similar to the design of FIGS. 1A, the thermally insulative housing and elongated internal chamber of FIG. 1B are designed to be affixed to the surgical scope tip to allow the surgical scope tip to be oriented freely, particularly in a side-ways manner, as well as upright or upside-down position, while inserted in the safety cap device.

In the example shown in FIG. 1B, the elongated internal chamber (e.g., 118) is disposed in the housing 106 and has a first end, a second end, and an internal surface defining a central internal volume to receive and retain a surgical scope tip. The first end includes one or more cover sealing members defining a device opening for receiving and conformally and frictionally sealing the surgical scope tip, and the second end is in contact with or proximal to the battery holder housing at least one battery. The elongated chamber can have a cross-section corresponding to the opening to define a gap to an exterior surface of the surgical scope tip when the surgical scope tip is disposed of in the internal volume. In some embodiments, the elongated internal chamber can be cylindrical. In some embodiments, the elongated internal chamber can have a shape in correspondence to the exterior surface of the surgical scope tip.

The heating element (e.g., heating coil, heating sheet, chopped transistor, traces on a printed circuit board (PCB), and others described herein) disposed on and in substantial thermal communication with an exterior surface of the elongated chamber for warming a liquid or sponge (not shown) disposed in the internal volume (substantial=spanning more than 50% of the area of the interior surface of the elongated chamber). In some embodiments, the heating element is encapsulated in a thermal insulating layer (e.g., insulating sheet or foam) in the internal volume. In other embodiments, the heating element may be placed at a bottom position and in contact with the elongated internal chamber.

The electric circuit is configured to be in electric communication with the heating element. In some embodiments, the electric circuit can comprise a thermoswitch 114 disposed on the surface of the heating element. The thermoswitch 114 is configured to break electrical contact between a battery (in the battery holder) and the heating element when the thermoswitch exceeds a predefined temperature threshold having a correspondence to a maximum temperature for the internal volume (e.g., between 50° C. and 55° C.). The thermoswitch is also configured to make electrical contact between the battery and the heating element when the surgical scope tip is disposed in the internal volume and in contact with the contact switch. In some embodiments, the electric circuit can comprise a voltage regulator to boost the voltage output of the battery (e.g., for rapid heating in combination with a thermally optimized heating chamber) and/or a toggle switch configured to make electrical contact between the battery and the heating element (e.g., an on/off switch 117).

The battery (in the battery holder) is sized with energy to rapidly heat the central internal volume in combination with heat from the surgical scope tip. In some embodiments, the battery (e.g., coin battery CR2477, CR123, or other standard battery form factor) used in the exemplary device can have a center axis corresponding to an elongated axis of the elongated thermally conductive chamber. In other embodiments, the battery can have a center axis perpendicular to an elongated axis of the elongated thermally conductive chamber. In other embodiments, the battery can have a center axis parallel to an elongated axis of the elongated internal chamber.

The thermally insulating housing can define (i) a first section to house the elongated thermally conductive chamber and heating element and (ii) a second section, as a battery holder, to house at least one battery, the second end of the elongated thermally conductive chamber in contact with a housing wall defined between the first section and the second section. In some embodiments, the thermally insulating housing can define (i) a bullet shape, (ii) a cylindrical shape, (iii) an inkwell shape, or (iv) a nose cone shape, as the internal volume and have a height to width (or diameter) ratio between 1:1 and 25:1 or between 1:1 and 5:1.

The cover sealing members may include a compliant cover sealing member that couples to an orifice defining the first end of the elongated thermally conductive chamber to define the device opening (e.g., the first compliant cover sealing member having a snap member that extends into a portion of the elongated thermally conductive chamber to couple therewith).

The cover sealing members may include a second compliant cover sealing member that couples to the first compliant cover sealing member, the first compliant cover sealing member, and the second compliant cover sealing member, each having a respective orifice to define the device opening. The first compliant cover sealing member can have a second orifice (e.g., to allow air venting during surgical scope tip insertion into the elongated thermally insulative chamber and through the first compliant cover sealing member).

Each sealing member can be a rubber seal configured to keep heat internal to the internal volume of the internal chamber after heating cycles. The internal volume of the internal chamber can be made of an insulating composition, e.g., plastic (<1 W/m·K) instead of stainless steel.

Example System #4. FIG. 1D shows an example safety cap for a surgical scope tip without a heating element comprising a thermally insulative housing and an elongated internal chamber. The internal surface 150 may be coated with a reflective coating or ceramic coating or may be fabricated to have a reflective surface.

In the example shown in FIG. 1D, the housing of the safety cap device defines (i) a first section to house the elongated internal chamber (e.g., a receptacle, as described in more detail with reference to FIG. 3A) and (ii) a second section, as a battery holder, to house at least one battery. In FIG. 1D, the exemplary safety cap employs the light source to provide passive heating (instead of active heating from a heating element in FIG. 1). In some embodiments, the vessel can have a reflectively-coated surface (e.g., electropolished coating with reflectivity >60%, mylar film with reflectivity >95%) to redirect light energy and create a warmer internal environment inside the internal chamber. In other embodiments, the vessel can have a ceramically coated surface with optically clear or reflective properties.

The internal chamber may include a liquid, foam, or gel to facilitate thermal energy retention and transfer to the surgical scope tip. In some embodiments, an insulating polyurethane foam lining having a thermal conductivity, e.g., between 0.02 and 0.03 W/(m·K) may be employed.

### Example Method

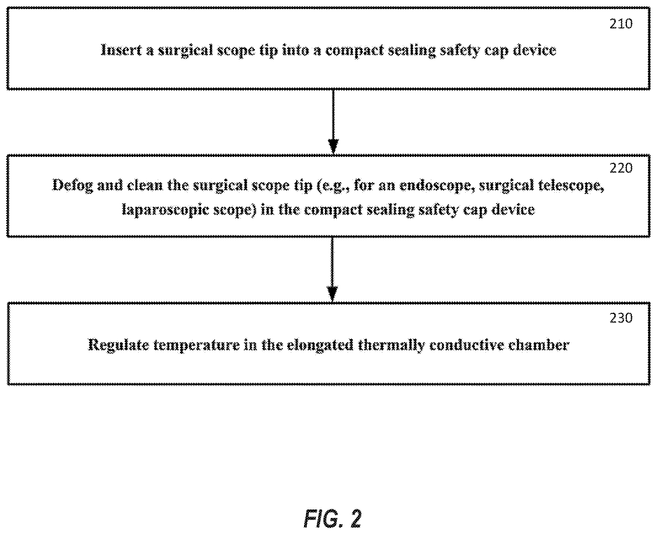

As shown in FIG. 2, at step 210, a surgical scope tip can be inserted into a compact sealing safety cap device. At step 220, the exemplary method can defog and clean a surgical scope tip (e.g., for an endoscope, surgical telescope, or laparoscopic scope) in the compact sealing safety cap device. At step 230, the exemplary method can regulate temperature in the elongated internal chamber. The exemplary safety cap device can be readily and beneficially placed over the surgical drape/covering and gowns over the patient, which would prevent the surgical scope tip from coming into contact with the surgical drape/covering and gowns. The method of FIG. 2 can be performed using any of the embodiments of the present disclosure described herein.

The compact form factor exemplary safety cap device also includes a thermally insulative housing and elongated internal chamber disposed in the housing having an internal surface defining a central internal volume to receive and retain a surgical scope tip and a heating element (e.g., heating coil, or heating sheet) disposed on and in substantial thermal communication (the term “substantial” here refers to the heating element spanning more than 50% of the area of the interior surface of the elongated chamber) with an interior surface of the elongated chamber for warming a liquid or sponge disposed in the internal volume to act as a scope warmer and prevent fogging of, and clean, the scope tips.

### Example Safety Cap

FIG. 1C shows an example safety cap configured with a housing member, a heating element, and a battery.

The housing member can enclose the surgical (robotic, endoscopic, laparoscopic, etc.) scope tip, providing a protective barrier between the high-energy output of the scope and flammable materials (e.g., surgical drape) present in the operating room (OR).

The heating element (shown as a heating element) can warm the scope tip to prevent fogging, ensuring clear visualization during surgery. The heating element is surrounded by an insulating material to help prevent heat loss. In some embodiments, the insulating material is in the form of a sheet or a foam.

A reservoir (not shown) contains a cleaning and/or defogging solution, which can remove contaminants (e.g., blood, tissue) on the scope tip. A sponge or foam material can also be disposed at the internal base of the reservoir.

An intelligent circuit (not shown) may be employed to sense the presence of a scope and rapidly heat the scope tip while managing power consumption over a long period of time (e.g., up to 40 degree c. in less than 5 minutes).

Characteristics of the exemplary safety cap. Table 1 shows example characteristics of the exemplary safety cap (e.g., 100).

TABLE 1

| Character- | |
|---|---|
| istics | Description |
| Rapid | When scope tips are housed in the exemplary safety cap, the |
| heating | scopes can be heated to an optimal temperature of 50 degrees |
| | Celsius, not to exceed 54.9 degrees Celsius. Various power |
| | configurations and heating element designs in the exemplary |
| | safety cap can provide a ramp-up heating time of less than |
| | 5 minutes. With circuit designs of the exemplary safety cap |
| | focused on a higher electrical current and an efficient |
| | heating element, the scope tip can reach the target temp in |
| | less than one minute. |
| Enhanced | The exemplary safety cap can shield the scope tip from |
| safety | accidental contact with flammable materials (e.g., surgical |
| | drapes, patient's skin), reducing the risk of fires |
| | and burns. |
| Compact | The exemplary safety cap's small footprint can provide |
| design | easy placement and positioning in the OR, maximizing scope |
| | utilization and minimizing disruption. Scopes are manipulated |
| | into various positions, and their safety cap should be minimal |
| | so as not to impede the activity of the surgical team. To |
| | accommodate this requirement, the exemplary safety cap is |
| | developed for compactness. An example Height is less than |
| | about 9 cm (e.g., about 8 cm), e.g., as an optimally |
| | ergonomic size, and the overall Height to Width (or |
| | Diameter) ratio is between 1.0:1 and 25:1 (e.g., between |
| | 1.3 and 1.4 or around 2.4). The ratio of the Width or |
| | Diameter is taken from the largest Width dimension of |
| | the exemplary safety cap. No Diameter or Width feature |
| | shall be larger than the Height of the exemplary safety cap. |
| Power | A built-in power management system can ensure rapid heating |
| Efficiency | while optimizing energy consumption, extending the device's |
| | operating life. The exemplary safety cap can be used without |
| | interruption for extended lengths of time (4+ hours) |
| | for more complex procedures. |
| Precise | The heating element can limit the temperature level below 50 |
| temper- | degrees Celsius, preventing overheating and damaging scopes, |
| ature | a common issue with existing products. Per the scope user |
| control | instructions, the temperature should not exceed 55 degrees |
| | Celsius. |

### Example System #4

Conventional receptacles for surgical scope tips (e.g., systems that protect and/or warm surgical scope tips) are generally heavy (e.g., greater than about 150 grams) and require that the surgical probe be held in a particular orientation to the receptacle to stay in the receptacle. Moreover, because conventional receptacles are heavy, they fall off the scope when the scope is picked up because the retaining force of the receptacle is less than the weight of the receptacle. These challenges limit the use of conventional receptacles for heating surgical scope tips and create ergonomic and practical limitations to their use in surgical procedures.

Embodiments of the present disclosure overcome these limitations of conventional receptacles. First, embodiments of the present disclosure include receptacles that have aspect ratios (ratios of length/height to horizontal width) that are optimized so that the receptacle can be tilted at any angle with the scope inserted. This overcomes the limitation of conventional receptacle shapes that require the scope to be held at a fixed angle relative to the receptacle. Tables 2 and 3 provide example mechanical retaining forces and size of the exemplary device. The example mechanical retaining force is at least an 8× retention-to-weight (e.g., for a ~50 gram surgical scope tip that provides, e.g., 8×-20×, e.g., for 5 mm, 8 mm, 10 mm sized scope tips).

TABLE 2
Scope Warmer Weight Comparison Table

| | Example Embodiment | Clearify | LaparoVue |
|---|---|---|---|
| | (e.g., FIG. 3) | #1 | #1 |
| Weight | 46 | 162 | 194 |
| (g) | | | |

TABLE 3
Retention Force of Example Embodiment

| Scope Ø | 5 mm | 8 mm | 10 mm |
|---|---|---|---|
| Force (g) | 400 | 580 | 640 |

Embodiments of the present disclosure include receptacles with reduced weight and/or increased retention force so that, when the scope is picked up, the receptacle remains attached to the scope. Example receptacles include optimized heating circuit design and control to reduce the number of batteries inside the receptacle and/or increased retaining force through one or more valve/gaskets. The receptacle can optionally be plastic (e.g., LDPE, HDPE) or metal (e.g., stainless steel). In some embodiments, heating features are integrated or embedded into the receptacle to improve heating. Alternatively or additionally, the receptacle can be formed as an integral structure with the valves or adhered to the valves.

Referring now to FIG. 3A, an example embodiment of the present disclosure can include a housing 300. The housing 300 can optionally be a thermally insulative housing. As used herein, the thermally insulative housing can be insulated by one or more air cavities in the housing 300, and/or one or more layers of foam. In some embodiments, the housing 300 can define a top surface 302, which can define a housing opening 306 configured to receive the surgical scope tip.

The housing 300 can be sized and shaped according to any of the example housing shapes described herein, for example, with reference to FIGS. 4-11. The housing 300 can be configured to be affixed to the surgical scope tip so that the surgical scope tip can be oriented freely while inserted in the device, including flat (e.g., parallel to a surface), or any tilted orientation position (e.g., at any angle to a surface). In some embodiments, the housing is sized to be provided as a compactly portable device and, optionally, can be designed for single use as a disposable device.

The housing 300 can be configured to receive a receptacle 320 configured to receive the surgical scope tip. The housing 300 can optionally include a first rib 308 and a second rib 310. A first valve seal 312 and a second valve seal 316 defining a second valve seal can be disposed between the first rib 308 and the second rib 310. Optionally, the first valve seal 312 and second valve seal 316 can be integrally formed into a single valve structure with two coaxial valve openings. Alternatively or additionally, embodiments of the present disclosure may include additional valve seals (not shown) coaxial with the first valve seal 312, or fewer valve seals (e.g., only the first valve seal 312).

Optionally, the first valve seal 312 defines a first valve seal opening 314, and the second valve seal 316 defines a second valve seal opening 318 as illustrated in FIG. 3B, which shows an exploded view of the first valve seal 312, the second valve seal 316, and the receptacle. Optionally, the first valve seal 312 and second valve seal 316 are configured to receive and retain probes with diameters between 5 mm and 12 mm. In some embodiments, the first valve seal opening 314, the second valve seal opening 318, and the housing opening 306 can be coaxial so that the surgical scope tip passes through the aligned opening region and into the device. As shown in FIG. 3B, the first valve seal 312 and the second valve seal 316 can optionally be different types of valve seals. For example, the second valve seal 316 can optionally be a slit-type or “duckbill” valve, as shown in FIG. 3B. In some embodiments, the valve seals 312, 316 can be compliant cover sealing members, silicone seals, rubber seals, or seals made of any other material. In some embodiments, one of the valve seals 312, 316 can be a Backup Seal (aka Lip Seal) and the other can be a Cross-Slit Valve, and the order of placement can be reversed.

As shown in FIG. 3A and FIG. 3B, the receptacle 320 can optionally hold a sponge 326. Optionally, the sponge can be treated with a foam, gel, or liquid. Alternatively or additionally, the sponge 326 can be sized (e.g., a certain height) to avoid rotating or translating within the receptacle. The sponge 326 can be configured to insulate the scope tip and/or retain fluid, and/or accommodate different angles of the scope tip (e.g., 0 to 70 degrees).

A circuit board 336 can be disposed opposite the receptacle opening 304 so that it contacts the closed side of the receptacle 320. Optionally, an adhesive (e.g., room-temperature-vulcanizing glue) can be used to improve thermal communication between the circuit board 336 and the receptacle. The circuit board 336 can be configured to implement any of the electric circuits and/or heating circuits described herein. In the example shown in FIG. 3A-3D, the circuit board is configured with a transistor 332 that is configured to generate heat that passes through a plurality of vias 340, as shown in the top-down view of the circuit board 336 in FIG. 3C. The circuit board 336 can optionally have a surface area generally conforming to a bottom portion of the receptacle 320 to improve heat transfer. For example, as shown in FIGS. 3A-3C, the receptacle 320 can optionally be cylindrical, and the circuit board 336 can also be approximately cylindrical. The circuit board 336 can include at least one thermal conducting via 340 and, in some embodiments, multiple thermal conducting vias 340 can collectively provide a thermally conductive pathway for thermal energy to travel through the circuit board 336 toward the receptacle 320 to heat the surgical scope tip. A transistor 332 can be coupled to the circuit board 336 and operatively coupled to a battery 334 to generate heat. In some embodiments, the circuit board 336 further includes a status LED 342 configured to indicate when the device is powered and/or heating.

The transistor 332 can optionally be configured to optimize the amount of heating efficiency of the system. In an example embodiment, the transistor is an NPN transistor. However, the size of the transistor may be limited by the size of the circuit board 336. In the example embodiment, the largest NPN transistor package size that would fit on the PCB was chosen. In alternative embodiments of the present disclosure, different sizes of transistors can be used.

Battery 334 can be disposed at a bottom portion inside the housing 300 opposite the housing opening and can be operatively coupled to the transistor 332. In some embodiments, the battery 334 can be a single battery (e.g., a CR123 or CR123A battery). Optionally, the battery 334 is a disposable lithium-ion battery.

The housing 300 can further define a bottom surface 344 configured to support the housing 300 in a vertical orientation. A vertical dimension 346 can be defined by a distance between the receptacle opening 304 and the bottom surface 344, and a horizontal dimension 348 can be perpendicular to the vertical dimension 350. In some embodiments, the vertical dimension 346 can be between 1.3 and 2.5 times larger than the horizontal dimension 348. More generally, the device can define a height-to-width (or diameter) ratio between 1:1 and 5:1 or between 1:1 and 25:1. The example embodiment illustrated in FIG. 3A is about 2.3.

The circuit board 336 can optionally include a thermal sensor 338 disposed on a second side of the printed board opposite the transistor, as shown in FIG. 3A. The thermal sensor 338 can be positioned at a side of, and in thermal contact with, the receptacle 320. The thermal sensor 338 can be a thermistor, thermoswitch, thermocouple, or any other type of thermal sensor. The thermal sensor 338 can optionally be positioned on an exterior side of the receptacle 320 and/or above the circuit board 336, so as to measure the temperature of the receptacle 320. The thermal sensor 338 can optionally be controlled by a control circuit 336, or by a microcontroller or other computing device. In the example shown in FIG. 3A, the thermal sensor 338 is a thermistor with a negative temperature coefficient and configured as part of an analog control circuit implemented on the circuit board 336.

In use, the surgical scope tip can be inserted through the housing opening 306, receptacle opening 304, the first valve seal opening 314, and the second valve seal opening 318, and into the receptacle 320. The valve seals 312, 316 can frictionally engage the surgical scope tip so that the device remains affixed to the surgical scope tip during handling outside of insertion in a human cavity (e.g., when the surgical scope tip is not being used to perform the procedure). In some embodiments, the valve seals 312, 316 can provide retention greater than the weight of the device and, in some examples, approximately 8-9 times retention force when compared to the weight of the device. The housing 300 can thereby enclose the surgical scope tip and provide a protective barrier between the high-energy output of the scope and surrounding thermally sensitive or flammable materials, while also supporting cleaning, defogging, and warming. The device can be orientation-agnostic while retaining the cleaning and/or defogging solution in the device.

Example configuration. FIGS. 3D-3F illustrate perspective exterior views of the embodiment of FIGS. 3A-3C. FIG. 3D illustrates a perspective view showing the first exterior side 351 of the housing 300. FIG. 3E illustrates a side view of the second exterior side 352. As shown in FIGS. 3D and 3E, optionally, both exterior sides include flattened sections. FIG. 3F illustrates a top view of the housing towards the housing opening, with the first valve seal opening also visible.

Alternative configurations. FIGS. 3G and 3H each illustrate an example implementation of the present disclosure, including a battery 334 and a receptacle 320. In FIG. 3G the battery 334 and receptacle 320 are mounted side-by-side inside a housing 300. While inserted in the safety cap device, having a compact internal housing that is small in size, lightweight, and having a height-to-width (or diameter) ratio between 1:1 and 5:1 that is ergonomically and optimally sized for a clinician's hand. FIG. 3G has a ratio between 1.3 and 1.4. In FIG. 3H, the battery 334 is mounted below the receptacle 320. FIG. 3H has a ratio of around 2.4. In FIG. 3G, the battery 334 has a center axis in parallel corresponding with the axis of the receptacle 320.

### Example Housing Configurations

FIGS. 4-9 and 11 illustrate alternative housing designs according to various embodiments of the present disclosure. The safety cap device defines a shape having a height-to-width (or diameter) ratio between 1:1 and 25:1, where the safety cap device is compactly portable when affixed to the surgical scope tip to allow the surgical scope tip to be oriented freely while inserted in the safety cap device. In some embodiments, the ratio is between 1.3 and 1.4 to be ergonomically and optimally sized for a clinician's hand. In some embodiments, the ratio is around 2.4 to be ergonomically and optimally sized for a clinician's hand. It should be understood that the housing designs are non-limiting example embodiments, and that different housing designs and features can be used in embodiments of the present disclosure. Additionally, the number, size, and model of batteries depicted and described as being used in the housings of FIGS. 4-9 and 11 are intended only as non-limiting examples, and any number of models of batteries can be used in various embodiments of the present disclosure.

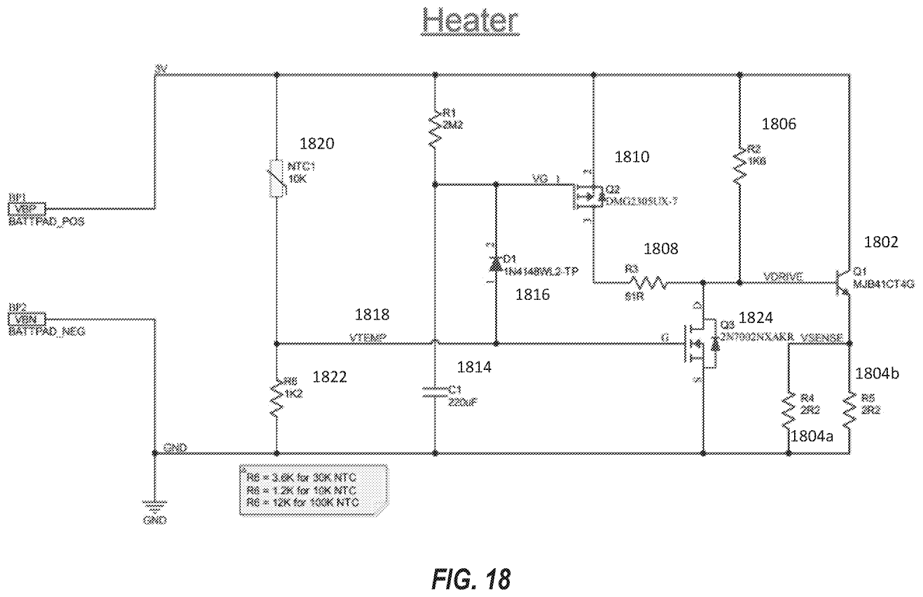

In combination with the fast ramping circuit described in relation to FIG. 1A and FIG. 18, the optimized electric heating circuit and controls can facilitate rapid heating of the internal components to ready it for use in less than 5 minutes, while in combination with an optimized thermal insulated structure, to operate at an optimal temperature, e.g., around 48-52° C., over an extended period, e.g., 4 hours, during a surgery, while drawing on a small battery (two coin cell batteries (e.g., CR2477-sized batteries).

The elongated internal chamber (e.g., 102) may be cylindrical, oval, or other receptacle-shaped. In some embodiments, the elongated internal chamber (e.g., 102) has a shape in correspondence to an exterior surface of the surgical scope tip.

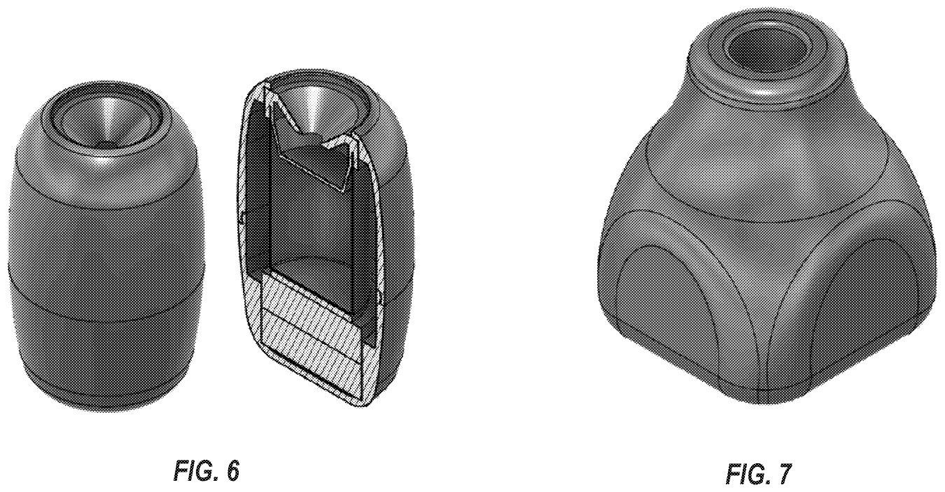

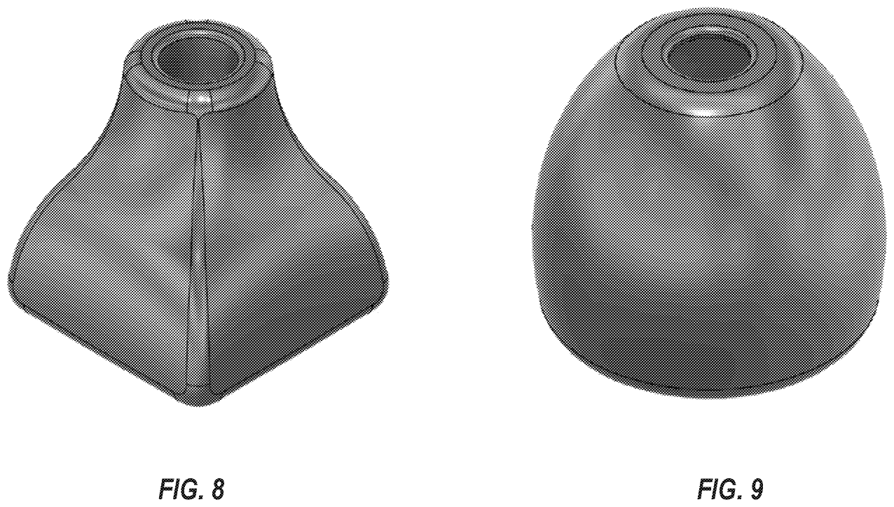

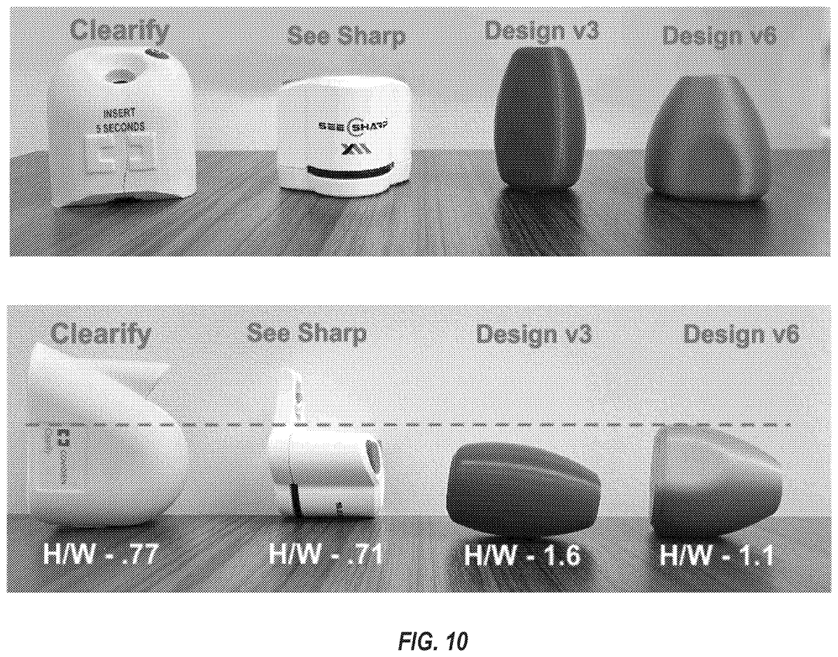

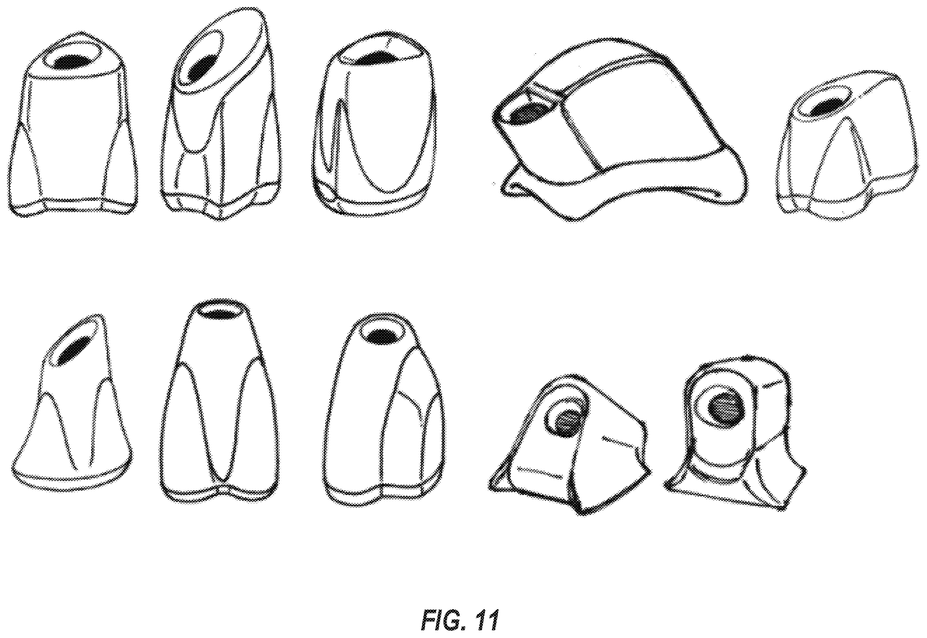

Specifically, FIG. 4 illustrates an example housing design configured to house two coin cell batteries (e.g., CR2477-sized batteries). FIG. 5 illustrates an example housing design with a bullet-shaped profile, and configured to house two batteries (e.g., CR123-sized batteries). FIG. 6 illustrates an example housing design configured to house two coin cell batteries (e.g., CR2477-sized batteries) with rounded edges. FIG. 7 illustrates an example housing design configured with an “inkwell” shape with rounded edges and configured to house two batteries (e.g., CR123-sized batteries). FIG. 8 illustrates another example housing design configured with an “inkwell” shape with rounded edges and configured to house two batteries (e.g., CR123-sized batteries). FIG. 9 illustrates an example housing with a nose-cone design. FIG. 10 illustrates an example embodiment of the present disclosure compared to other conventional devices.

### Example Method of Operation

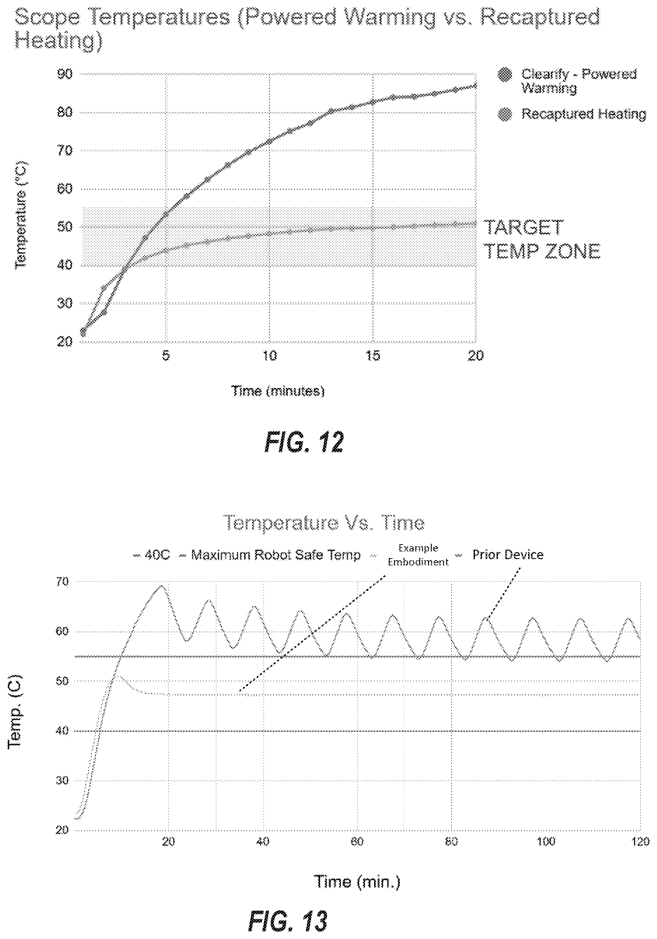

FIG. 12 illustrates a plot of the temperature of the surgical scope tip in (i) active heating (i.e., powered warming) using a heat element and (ii) passive heating (i.e., recaptured heating). FIG. 12 shows a temperature profile as a target temperature zone is sufficient to keep the surgical tip from fogging while minimizing the energy use to allow for extended operation to be used via a small battery. The optimized temperature profile and operation of the circuit and thermal insulated structure were observed to extend the operation of the exemplary safety cap device and method by over 2× over initial baseline designs without such optimizations (e.g., in the circuit, component selection).

FIG. 13 illustrates a plot of the thermal stability of an example embodiment of the present disclosure vs. a conventional scope warmer and the maximum safe temperature of an example surgical scope tip. As shown in FIG. 13, the example embodiment is shown configured to heat up the internal chamber (e.g., 102) to over 40° C. in about or less than 5 minutes. There is a slight overshoot to about 50° C., and the chamber is allowed to cool to about 47° C. and 48° C., which is maintained steady by constant modulated control of a heating transistor and resistor(s).

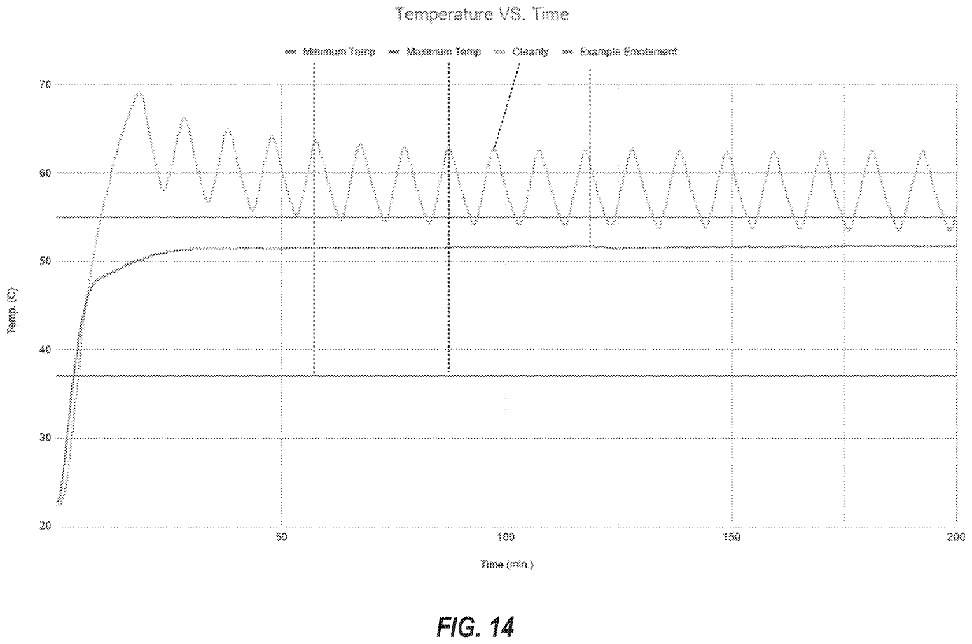

FIG. 14 illustrates another plot of temperature vs. time for the example embodiment of the present disclosure compared to a conventional scope warmer and the maximum safe temperature of an example surgical scope tip. The current state-of-the-art products on the market are from Covidien (e.g., Clearify) and Medix3d LLC (e.g., See Sharp). Conventional devices can be bulky, cause excessive heating, be excessively large, and/or can be cumbersome. Its thermal heating profile is similar to that of Covidien's Clearify, but does not exceed the 55 degrees Celsius heating threshold.

### Example Seal Configurations

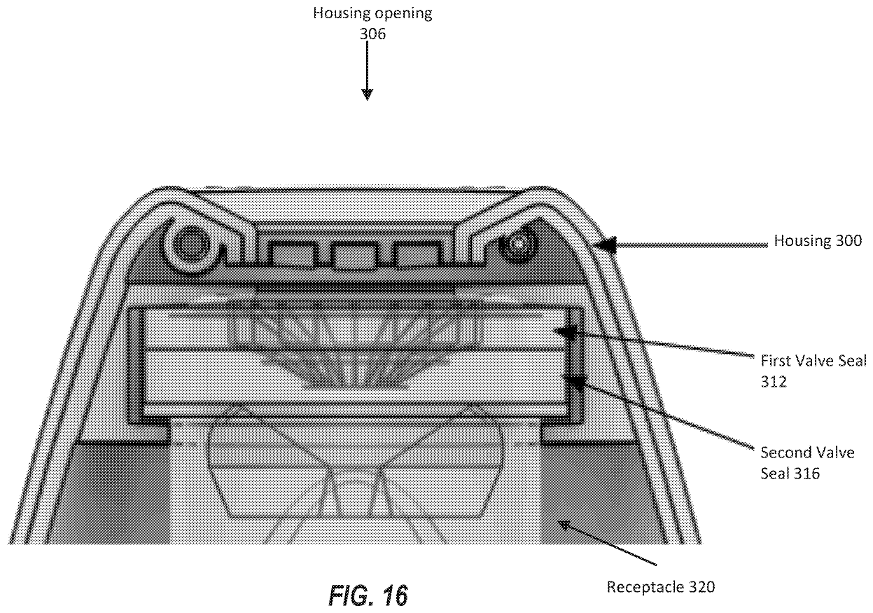

FIG. 16 illustrates an enlarged view of the housing opening 306, housing 300, first valve seal 312, and second valve seal 316 according to implementations of the present disclosure. The first rib 308 and the second rib 310 can be configured to retain the first valve seal 312 and the second valve seal 316 and to apply compression to the valve seals 312, 316 against a retaining lip 322 of the receptacle.

The first valve seal 312 may define a first valve seal opening 314, and the second valve seal 316 defines a second valve seal opening 318, as illustrated in FIG. 3B, which shows an exploded view of the first valve seal 312, the second valve seal 316, and the receptacle. The first valve seal 312 and second valve seal 316 may be configured to receive and retain probes with diameters, e.g., between 5 mm and 12 mm. In some embodiments, the first valve seal opening 314, the second valve seal opening 318, and the housing opening 306 can be coaxial so that the surgical scope tip passes through the aligned opening region and into the device. As shown in FIG. 3B, the first valve seal 312 and the second valve seal 316 can optionally be different types of valve seals. For example, the second valve seal 316 can optionally be a slit-type or “duckbill” valve, as shown in FIG. 3B. In some embodiments, the valve seals 312, 316 can be compliant cover sealing members, silicone seals, rubber seals, or seals made of any other material. In some embodiments, one of the valve seals 312, 316 can be a Backup Seal (aka Lip Seal) and the other can be a Cross-Slit Valve, and the order of placement can be reversed.

The valve seals 312, 316 may be assembled from individual seals to form an assembly, e.g., where the valve seals are adhered to one another as a unitary structure.

### Example Printed Electric Circuit

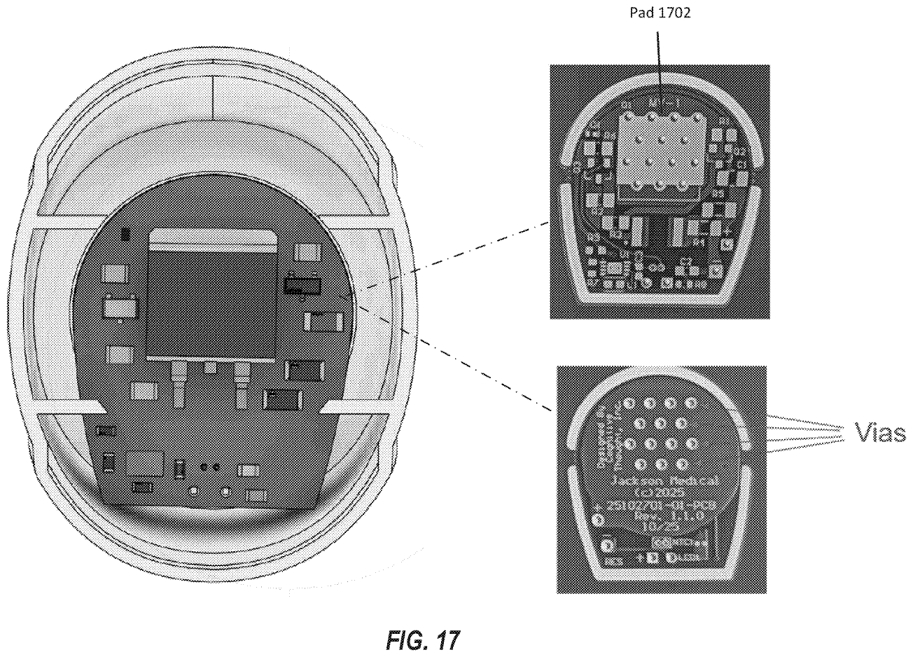

FIG. 17 illustrates a non-limiting example circuit design configured for a form factor capable of multiple orientations, according to implementations of the present disclosure. In FIG. 17, the printed circuit board includes a pad 1702 for placement of a heating transistor (e.g., Q1). The pad 1702 may include vias or thermal conducting structures to direct heat generated on the pad side to the other side of the printed circuit board, where the internal chamber 102 is positioned.

### Example Fast Ramp-Up Circuit with Long Duration Operation

FIG. 18 illustrates an example circuit design 118 (shown as 1800) for fast ramping of temperature up to a desired temperature for scope warming, according to implementations of the present disclosure. The electric circuit 118 may be configured with low voltage optimization and operation, which allows for the longer duration operation (while also having sufficient ramp-up time.

The electric circuit 1800 includes (i) a transistor and (ii) a thermal sensor, wherein the thermal sensor operatively couples, as a voltage divider circuit, to the transistor to drive the modulation of the transistor at low voltage at less 3.3V, wherein modulation or current flow through the transistor generates heat, and wherein the thermal sensor in the voltage divider circuit provide feedback control to the transistor to maintain a temperature of the thermal sensor (or the elongated internal chamber) at a pre-defined temperature.

The circuit 1800 may be powered from a single CR123A battery, which can serve as the power supply for the heating. The CR123A battery is rated at 3V and approximately 1500 mAh. Other voltage and energy storage may be used at a trade-off for size and weight. The mAh rating of the CR123A is vendor-dependent and, in most cases, it is around 1500 mAh.

Fast Temperature Ramp-Up Circuit. In FIG. 18, Transistor Q1 (1802) is a switching element configured to drive a load to ground to cause the circuit and internal structure to heat up. Transistor Q1 (1802) may be an NPN Transistor (e.g., current-controlled transistor). The package size and thermal heat sink plate of transistor Q1 (1802) may be selected to allow Q1 (1802) to heat up the PCB quickly. The PCB where Q1 (1802) is positioned may include a number of vias or thermal conductive channels to allow the heat from Q1 (1802) to pass into the PCB and allow the PCB to heat up. The transistor Q1 (1802) may drive a load (e.g., 1.1-ohm load, e.g., comprising two 2.2 ohm resistors in parallel circuit (shown as “R4” 1804a and “R5” 1804b). The multiple-resistor in parallel circuit configuration can allow for fast temperature dissipation across the resistors. A larger resistor with a higher wattage rating could be used, though size considerations may be taken into account. The values of R4 and R5 can be changed to control the maximum and minimum heater temperature ranges (heater current flow).

As shown in FIG. 18, Q1's base is pulled to 3V via resistor R2 (1806), wherein R2 (1806) sets the operational current (or holding temperature) of the heater for the life of the heater. Increasing or decreasing the value of R2 (1806) can adjust the baseline temperature of the heater after the heater temperature ramp has stopped. R2 (1806) and R3 (1808) (collectively, as VDRIVE) are in parallel to each other. The resistor R3 (1808) is connected to 3V via transistor Q2 (1810). The transistor Q2 (1810) may be a PMOSFET Transistor. The Q2's gate may be controlled via a resistor R1 (1812) and a capacitor C1 (1814).

At power up, the capacitor C1 (1814) can charge to the bus voltage of 3V via the resistor R1 (1812). While C1 (1814) is charging, it can keep the gate (shown as “vg”) of Q2 (1810) low. While Q2's gate “vg” is low, Q2 (1810) is turned on, allowing R3 (1808) to be connected to 3V. While R3 (1808) is connected to 3V, it draws current into the base (shown as VDRIVE) of Q1 (1802). The draw of current into the base of Q1 (1802) causes Q1 (1802) to heat up quickly (to provide the temperature ramp), so that the heater temperature holding set point can be reached faster. Once C1 (1814) has charged up, Q2 (1810) is turned off, which stops R3 (1808) from drawing current into Q1's base (VDRIVE), thus stopping the heating. The ramp-up profile can be adjusted by changing R1 (1812), C1 (1814), and R3 (1808) to increase or decrease the rate of temperature ramping profile. The resistor R1 (1812) and C1 (1814) thus control the “on” time, and R3 (1803) controls the maximum or minimum temperature.

A Zener diode D1 (1816) may be added to accelerate the charging of C1 (1814), where D1 (1816) may be driven from VTEMP (1818). As the temperature increases, VTEMP voltage can increase, so D1 (1816) can provide additional current from VTEMP (1818) to charge C1 (1814), so that C1 (1814) can stop charging more quickly once the set temperature has been reached. Thermal sensor NTC1 (1820) and resistor R6 (1822) form a voltage divider circuit. As the temperature impacting the thermal sensor NTC1 (1820) increases, the resistance of NTC1 (1820) can decrease. As the NTC1 (1820) resistance lowers, the voltage at the junction of NTC1 (1820) and R6 (1822) can increase at VTEMP (1818), where VTEMP (1818) is connected to the gate of Q3 (1824), an NMOSFET transistor. The increase in VTEMP voltage can cause Q3 (1824) to conduct, which allows Q3 (1824) to draw current away from the base Q1 (1802) (at VDRIVE), which can cause Q1 (1802) to lower its current draw/heater temperature. The thermal sensor NTC1 (1820) may be a 10K device, but could be a 100K, a 30K device, among others. The resistor R6 (1822) with NTC1 (1820) can control the temperature of the system. Changing the value of R6 (1822) can push up or down the temperature band of operation of the circuit.

Voltage generator for LED life indication. Because the battery voltage is only 3V for the heater and the heater loads down the battery to approximately 2.7V during heating, a boost voltage generator may be employed to power an LED, indicating the operation and life of the heater. The boost voltage generator may produce 5V for the LED circuit to provide a forward voltage of 2.8V to the LED. The voltage boost generator ensures the LED can operate throughout the operation of the header. Without the generator, the LED may illuminate for only a portion of the operation of the heater operation. With the voltage boost generator, the LED is currently lit longer than the heater can produce useful heat. In some embodiments, the life of the heater is determined as when the CR123A battery can supply sufficient current to keep the heater operational. The electric circuit 1800 may operate without an on/off switch, and may be initiated by pulling an electric insulating element positioned between the battery and the battery contact to allow the battery to make electric contact with the electric circuit. The LED provides a warming indication for the device with purely analog circuitry (without the need for a processor).

It is contemplated, in some embodiments, that Q1 (1802), Q2 (1810), and Q3 (1824) could be NMOSFET, PMOSFET, PNP or NPN Transistors. In FIG. 18, Q1 (1802) is a NPN Transistor, Q2 (1810) is a PMOSFET, and Q3 (1824) is an NMOSFET.

Transistor Selection Consideration. The size of the PCB may define the size of the side of the NPN transistor that can be used. The exemplary system may employ the largest NPN transistor package size that would fit on the PCB chosen. If more PCB space were available, a larger package size could be used. A package with a smaller size package may not produce enough body heat, which is used in part for the heater. So PCB size and the transistor package body may define the size of the design.

In some embodiments, the transistor (e.g., Q1) has a wattage rating of 65 Watts. A higher wattage transistor may have a larger thermal pad. The example embodiment uses the transistor thermal pad as the heating source. Tⱼ (thermal junction temperature) should be high enough for the transistor to operate above the heater target point. The Tⱼ of the example Transistor is around 150° C. For the thermal Resistance (junction to case and junction to Ambient), the lower the thermal resistance, the more heat is moved off the Transistor (junction) and into the PCB for the heater. For the NPN Transistor, hfe (gain) can also be used as a design factor. A higher hfe means a lower base voltage can produce a higher collector/emitter current, which increases self-heating of the device in the heater circuit. The NPN Transistor is currently used in the heater. The hfe increases with an increase in the transistor's ambient temperature.

Optionally, a MOSFET transistor can be used in place of the NPN transistor described herein. For example, the NPN transistor may be as good or better in lower voltage applications like the heater when the power source is only 3V. However, as described herein, the present disclosure contemplates different voltages of battery and different numbers of batteries, so that a MOSFET may improve over the NPN transistor for higher voltage applications.

### Discussion

The exemplary safety cap provides a solution for enhancing safety and efficiency in operating rooms (ORs). Configured to prevent fires and burns, the exemplary safety cap can be a protective barrier between high-energy scope tips and potential fuel sources. The exemplary safety cap tip can also function as a scope warmer, preventing fogging and ensuring clear visualization during surgery.

Unlike bulky and cumbersome state-of-the-art technologies, the exemplary safety cap can insulate the high-energy scope tip from potential fuel sources in the OR, reducing the risk of fires and burns.

An aspect of the exemplary safety cap is configuring the power and heating system to enable heating of the scope tip to its target temperature in under a minute, compared to the 5-minute heating time of existing products on the market. This rapid heating may translate to substantial time savings in the OR, where time is often measured in dollars. Compared to state-of-the-art systems, the exemplary safety cap's ability to heat scopes in less than 1 minute can save hospitals $60 per minute in the OR, saving $240 per surgery for the hospital, in one example scenario.

The exemplary safety cap can minimize waste and optimize storage space in hospitals. By reducing the safety cap's overall size, hospitals can store up to ten times the number of units compared to products available on the market, which can enhance operational efficiency and reduce costs associated with product procurement and storage. The exemplary safety cap can have a packaging volumetric footprint in the range of 12 in³ to 60 in³, while the current state-of-the-art systems have packaging volumes of 155 in³ (e.g., Covidien's Clearify) and 68 in³ (e.g., Xodus Medical's See Sharp). Moreover, the exemplary safety cap's smaller footprint and lightweight design make it easier to handle and use, improving workflow and reducing the risk of accidental damage.

### CONCLUSION

The construction and arrangement of the systems and methods, as shown in the various embodiments, are illustrative only. Although only a few embodiments have been described in detail in this disclosure, many modifications are possible (e.g., variations in sizes, dimensions, structures, shapes, proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions, and arrangement of the embodiments without departing from the scope of the present disclosure.

Although the figures show a specific order of method steps, the order of the steps may differ from what is depicted. Also, three or more steps may be performed concurrently or with partial concurrence. Such variation will depend on the software and hardware systems chosen and on the designer's choice. All such variations are within the scope of the disclosure. Likewise, software embodiments could be accomplished with programming techniques with rule-based logic and other logic to accomplish the various connection steps, processing steps, comparison steps, and decision steps.

It is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense but for explanatory purposes.

Disclosed are components that can be used to perform the disclosed methods and systems. These and other components are disclosed herein, and it is understood that when combinations, subsets, interactions, groups, etc. of these components are disclosed while specific reference of each various individual and collective combinations and permutation of these may not be explicitly disclosed, each is specifically contemplated and described herein, for all methods and systems. This applies to all aspects of this application, including, but not limited to, steps in disclosed methods. Thus, if there are a variety of additional steps that can be performed it is understood that each of these additional steps can be performed with any specific embodiment or combination of embodiments of the disclosed methods.

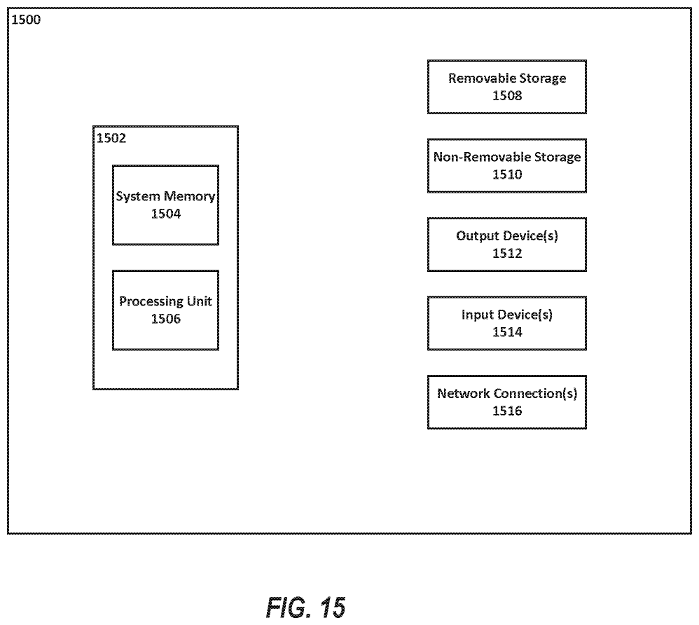

Referring to FIG. 15, an example computing device 1500 upon which the methods described herein may be implemented is illustrated. It should be understood that the example computing device 1500 is only one example of a suitable computing environment upon which the methods described herein may be implemented. Optionally, the computing device 1500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 1500 typically includes at least one processing unit 1506 and system memory 1504. Depending on the exact configuration and type of computing device, system memory 1504 may be volatile (such as random access memory (RAM)), non-volatile (such as read-only memory (ROM), flash memory, etc.), or some combination of the two. This most basic configuration is illustrated in FIG. 15 by box 1502. The processing unit 1506 may be a standard programmable processor that performs arithmetic and logic operations necessary for operation of the computing device 1500. The computing device 1500 may also include a bus or other communication mechanism for communicating information among various components of the computing device 1500.

Computing device 1500 may have additional features/functionality. For example, computing device 1500 may include additional storage such as removable storage 1508 and non-removable storage 1510 including, but not limited to, magnetic or optical disks or tapes. Computing device 1500 may also contain network connection(s) 1516 that allow the device to communicate with other devices. Computing device 1500 may also have input device(s) 1514 such as a keyboard, mouse, touch screen, etc. Output device(s) 1512 such as a display, speakers, printer, etc. may also be included. The additional devices may be connected to the bus in order to facilitate communication of data among the components of the computing device 1500. All these devices are well known in the art and need not be discussed at length here.

The processing unit 1506 may be configured to execute program code encoded in tangible, computer-readable media. Tangible, computer-readable media refers to any media that is capable of providing data that causes the computing device 1500 (i.e., a machine) to operate in a particular fashion. Various computer-readable media may be utilized to provide instructions to the processing unit 1506 for execution. Example tangible, computer-readable media may include, but is not limited to, volatile media, non-volatile media, removable media and non-removable media implemented in any method or technology for storage of information such as computer readable instructions, data structures, program modules or other data. System memory 1504, removable storage 1508, and non-removable storage 1510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example embodiment, the processing unit 1506 may execute program code stored in the system memory 1504. For example, the bus may carry data to the system memory 1504, from which the processing unit 1506 receives and executes instructions. The data received by the system memory 1504 may optionally be stored on the removable storage 1508 or the non-removable storage 1510 before or after execution by the processing unit 1506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware embodiments.

It is contemplated that features of the following patents, applications, and publications, as listed below and throughout this document, may be implemented in combination with the electrical, mechanical, material, or design feature provided herein, the following are hereby incorporated by reference.
U.S. Pat. No. 9,526,409 (issued Dec. 27, 2016).
U.S. Pat. No. 10,617,289 (issued Apr. 14, 2020).
U.S. Pat. No. 11,284,790 (issued Mar. 29, 2022).
U.S. Pat. No. 10,080,488 (issued Sep. 25, 2018).
U.S. Pat. No. 10,575,722 (issued Mar. 3, 2020).
U.S. Pat. No. 10,881,284 (issued Jan. 5, 2021).
U.S. Pat. No. 10,939,812 (issued Mar. 9, 2021).
U.S. Pat. No. 11,266,306 (issued Mar. 8, 2022).
U.S. Patent Application No. 2021/0085167 A1 (published Mar. 25, 2021).

What is claimed:

1. A device for a surgical scope tip comprising:
a thermally insulative housing defining a receptacle opening on a top surface and configured to receive a surgical scope tip;
an elongated internal chamber disposed in the housing and having a first end, a second end, and an internal surface defining a central internal volume to receive and retain a surgical scope tip for protection against thermal damage to surrounding thermally sensitive materials;
a circuit board or circuit component disposed to a surface of the elongated internal chamber distal and below the receptacle opening; and
a transistor coupled to the circuit board or circuit component and operatively coupled to a battery to generate heat (e.g., wherein the transistor is configured to heat the receptacle through the at least one thermal via),
wherein the device defines a shape having a height-to-width ratio between 1:1 to 5:1 and wherein the device is compactly portable when affixed to the surgical scope tip to allow the surgical scope tip to be oriented freely while inserted in the safety cap device.

2. The device of claim 1, wherein the circuit board or circuit component includes a sensor disposed on a second side of the printed board, wherein the sensor is positioned at a side of, to be in thermal contact with, the elongated internal chamber.

3. The device of claim 1, wherein the circuit board or circuit component has a surface area generally conforming to a bottom portion of the elongated internal chamber.

4. A device for a surgical scope tip comprising:
a first valve seal defining a first valve seal opening;
a second valve seal defining a second valve seal opening; and
a housing defining a housing opening having a first rib and a second rib, wherein the first and second rib are configured to retain the first valve seal and second valve seal;
wherein the first valve seal and second valve seal are disposed between the first rib and the second rib so that the first valve seal opening, second valve seal opening, and housing opening are substantially coaxial, and wherein the first and second rib are configured to apply compression to the first valve seal and the second valve seal,
wherein the device defines a shape having a height-to-width ratio between 1:1 and 5:1, and wherein the device is compactly portable when affixed to the surgical scope tip to allow the surgical scope tip to be oriented freely while inserted in the safety cap device.

5. The device of claim 4, further comprising a receptacle with a first end and a second end, wherein the first end comprises an opening and a retaining lip, and wherein the retaining lip is configured to be positioned between the first rib and second rib of the housing (e.g., wherein the first valve seal and the second valve seal are configured to provide at least 8× retention to the weight of the device (e.g., 50 grams) (e.g., 8××20×, e.g., for 5 mm, 8 mm, 10 mm sized scope tips)

6. The device of claim 4 or 5 further comprising:
an elongated internal chamber disposed in the housing and having a first end, a second end, and an internal surface defining a central internal volume to receive and retain a surgical scope tip for protection against thermal damage to surrounding thermally sensitive materials; and
a circuit board or circuit component disposed to (or around) a bottom surface of the elongated internal chamber distal and below the receptacle opening, wherein the circuit board or circuit component comprises a transistor coupled to the circuit board or circuit component and operatively coupled to a battery to generate heat.

7. A device for a surgical scope tip comprising:
a housing defining a receptacle opening configured to receive a surgical scope tip, and a bottom surface configured to support the housing in a vertical orientation;
wherein the housing defines a vertical dimension and a horizontal dimension, wherein the vertical dimension is defined by a distance between the opening and the bottom surface, and wherein the horizontal dimension is perpendicular to the vertical dimension, and wherein the vertical dimension is between 1.3 and 2.4 times larger than the horizontal dimension; and
a battery disposed at a bottom portion inside the housing.

8. A device for a surgical scope tip (e.g., for an endoscope, surgical telescope, laparoscopic, robotic scope) comprising:
an electric circuit disclosed on a surface of the elongated internal chamber, the electric circuit comprising (i) a transistor and (ii) a thermal sensor, wherein the thermal sensor operatively couples, as a voltage divider circuit, to the transistor to drive the modulation of the transistor at low voltage at less 3.3V, wherein modulation or current flow through the transistor generates heat, and wherein the thermal sensor in the voltage divider circuit provide feedback control to the transistor to maintain a temperature of the thermal sensor at a pre-defined temperature.

9. A device for a surgical scope tip comprising:
a thermally insulative housing; and
an elongated internal chamber disposed in the housing and having a first end, a second end, and an internal surface defining a central internal volume to receive and retain a surgical scope tip for protection against thermal damage to surrounding thermally sensitive materials,
wherein the safety cap device defines a shape having a height-to-width (or diameter) ratio between 1:1 and 5:1, and wherein the safety cap device is compactly portable when affixed to the surgical scope tip to allow the surgical scope tip to be oriented freely while inserted in the safety cap device.

10. The device of claim 9, further comprising:
a heating element disposed on and in substantial thermal communication with an exterior surface of the elongated chamber for warming a liquid or sponge disposed in the internal volume.

11. The device of claim 9 or 10, further comprising:
an electric circuit in electric communication with the heating element,
wherein the first end comprises one or more cover sealing members defining a device opening to receive and seal the surgical scope tip, wherein the second end is in contact with, proximal to, or surrounded by a battery holder housing at least one battery, and wherein the elongated chamber has a cross-section corresponding to the opening to define a gap to an exterior surface of the surgical scope tip when the surgical scope tip is disposed in the internal volume.

12. The device of any one of claims 9-11, wherein the heating element is encapsulated in a thermal insulating layer in the internal volume

13. The device of any one of claims 9-12, further comprising:
an electric circuit disposed in the central internal volume of the internal chamber,
wherein the first end comprises one or more cover sealing members defining a device opening to receive and seal the surgical scope tip, wherein the second end is in contact with, proximal to, or surrounded by a battery holder housing at least one battery, and wherein the elongated chamber has a cross-section corresponding to the opening to define a gap to an exterior surface of the surgical scope tip when the surgical scope tip is disposed in the internal volume.

14. The device of any one of claims 9-13, wherein the housing defines (i) a first section to house the elongated internal chamber and heating element and (ii) a second section, as a battery holder, to house the at least one battery, the second end of the elongated internal chamber in contact with a housing wall defined between the first section and the second section.

15. The device of any one of claims 9-14, wherein the housing defines (i) a first section to house the elongated internal chamber and (ii) a second section, as a battery holder, to house the at least one battery, the second end of the elongated internal chamber in contact with a housing wall defined between the first section and the second section.

16. The safety cap device of any one of claims 1-15, wherein the elongated internal chamber is cylindrical or oval.

17. The safety cap device of any one of claims 1-16, wherein the elongated internal chamber has a shape in correspondence to an exterior surface of the surgical scope tip.

18. The safety cap device of any one of claims 1-17, wherein the battery has a center axis corresponding to an elongated axis of the elongated internal chamber.

19. The safety cap device of any one of claims 1-17, wherein the battery has a center axis perpendicular to an elongated axis of the elongated internal chamber.

20. The safety cap device of any one of claims 1-19, wherein the battery has a center axis parallel to an elongated axis of the elongated internal chamber.

21. The safety cap device of any one of claims 1-20, wherein the battery is disposed proximal to the surgical scope tip.

22. The safety cap device of any one of claims 1-21, wherein the external housing has (i) a bullet shape, (ii) a cylindrical shape, (iii) an inkwell shape, or (iv) a nose cone shape having a height-to-width (or diameter) ratio between 1:1 and 5:1.

23. The device of any one of claims 4-22, wherein the electric circuit comprises a thermistor disposed on a surface of the heating element.

24. The device of any one of claims 4-23, wherein the electric circuit comprises a thermistor configured to break electrical contact between the battery and the heating element when the thermoswitch exceeds a predefined temperature threshold having a correspondence to a maximum temperature for the internal volume.

25. The device of any one of claims 1-22, wherein the electric circuit comprises a thermoswitch or thermocouple configured to control the heating element.

26. The device of any one of claims 1-25, wherein the electric circuit comprises a contact switch configured to make electrical contact between the battery and the heating element when the surgical scope tip is disposed in the internal volume and in contact with the contact switch.

27. The device of any one of claims 1-26, wherein the electric circuit comprises a toggle, pull tab or switch configured to make electrical contact between the battery and the heating element.

28. The device of any one of claims 1-27, wherein the electric circuit comprises a voltage regulator to boost the voltage output of the battery.

29. The device of any one of claims 1-28, wherein the battery is sized with energy to rapidly (e.g., within 4-5 minutes) heat the central internal volume in combination with heat from the surgical scope tip.

30. The device of any one of claims 1-29, wherein the one or more cover sealing members includes a first compliant cover sealing member that couples to an orifice defining the first end of the elongated internal chamber to define the device opening (e.g., the first compliant cover sealing member having a snap member that extends into a portion of the elongated internal chamber to couple therewith).

31. The device of any one of claims 1-30, wherein the one or more cover sealing members includes a second compliant cover sealing member that couples to the first compliant cover sealing member, the first compliant cover sealing member and the second compliant cover sealing member each having a respective orifice to define the device opening.

32. The device of any one of claims 1-31, wherein the first compliant cover sealing member has a second orifice (e.g., to allow air venting during surgical scope tip insertion into the elongated internal chamber and through the first compliant cover sealing member).

33. A method comprising:
inserting a surgical scope tip into a housing of a device according to any one of claims 1-32;
defogging or cleaning the surgical scope tip; and
regulating temperature in the receptacle.

34. The method of claim 33, wherein cleaning the surgical scope tip comprises applying a foam to the surgical scope tip.

35. The method of claim 33, wherein the device comprises a vent aperture and inserting the surgical scope tip comprises venting air through the vent aperture.
